(19) United States
(12) Patent Application Publication
(10) Pub. No.: US 2013/0095076 A1
(43) Pub. Date: Apr. 18, 2013

(54) Methods For Increasing Blood Flow And/Or Promoting Tissue Regeneration

(71) Applicants: Gary J. BRIDGER, Bellingham, WA (US); Simon P. FRICKER, Langley (CA)

(72) Inventors: Gary J. BRIDGER, Bellingham, WA (US); Simon P. FRICKER, Langley (CA)

(73) Assignee: Genzyme Corporation, Cambridge, MA (US)

(21) Appl. No.: 13/542,612

(22) Filed: Jul. 5, 2012

Related U.S. Application Data
(63) Continuation of application No. 12/280,542, filed on Jan. 26, 2009, now abandoned.

(51) Int. Cl. A61K 35/54

(52) U.S. Cl. CPC A61K 35/545
USPC 424/93.7; 540/474

(57) ABSTRACT

Methods for increasing blood flow and/or regenerating tissue using compounds which bind to the chemokine receptor CXCR4 are disclosed. Preferred embodiments of such compounds are of the formula

Z-linker-Z′ (1).

### RELATED APPLICATION(S)

This application is a continuation of U.S. application Ser. No. 12/280,542, filed Jan. 26, 2009, which is the U.S. National Stage of International Application No. PCT/US2006/006435, filed on Feb. 24, 2006, published in English.

The entire teachings of the above application(s) are incorporated herein by reference.

### TECHNICAL FIELD

The invention is in the field of therapeutics and medicinal chemistry. More particularly, the invention concerns methods for increasing blood flow and/or promoting tissue regeneration.

### BACKGROUND ART

Blood cells play a crucial part in maintaining the health and viability of animals, including humans. White blood cells include neutrophils, macrophage, eosinophils and basophils/mast cells as well the B and T cells of the immune system. White blood cells are continuously replaced via the hematopoietic system, by the action of colony stimulating factors (CSF) and various cytokines on stem cells and progenitor cells in hematopoietic tissues. The nucleotide sequences encoding a number of these growth factors have been cloned and sequenced. Perhaps the most widely known of these is granulocyte colony stimulating factor (G-CSF) which has been approved for use in counteracting the negative effects of chemotherapy by stimulating the production of white blood cells and progenitor cells (peripheral blood stem cell mobilization). A discussion of the hematopoietic effects of this factor can be found, for example, in U.S. Pat. No. 5,582,823, incorporated herein by reference.

Several other factors have been reported to increase white blood cells and progenitor cells in both human and animal subjects. These agents include granulocyte-macrophage colony stimulating factor (GM-CSF), Interleukin-1 (IL-1), Interleukin-3 (IL-3), Interleukin-8 (IL-8), PIXY-321 (GM-CSF/IL-3 fusion protein), macrophage inflammatory protein, stem cell factor, thrombopoietin and growth related oncogene, as single agents or in combination (Dale, D., et al., Am. J. of Hematol. (1998) 57:7-15; Rosenfeld, C., et al., Bone Marrow Transplantation (1997) 17:179-183; Pruijt, J., et al., Cur. Op. in Hematol. (1999) 6:152-158; Broxmeyer, H., et al., Exp. Hematol. (1995) 23:335-340; Broxmeyer, et al., Blood Cells, Molecules and Diseases (1998) 24:14-30; Glaspy, J., et al., Cancer Chemother. Pharmacol. (1996) 38 (suppl): S53-S57; Vadhan-Raj, S., et al., Ann. Intern. Med. (1997) 126:673-81; King, A., et al., Blood (2001) 97:1534-1542; Glaspy, J., et al., Blood (1997) 90:2939-2951).

While endogenous growth factors are pharmacologically effective, the well known disadvantages of employing proteins and peptides as pharmaceuticals underlies the need to add to the repertoire of such growth factors with agents that are small molecules. In another aspect, such small molecules are advantageous over proteins and peptides where production in large quantities are desired.

A number of cyclic polyamine antiviral agents have been described in a series of U.S. patents and applications (See e.g., U.S. Pat. Nos. 5,021,409; 6,001,826; 5,583,131; 5,698,546; and 5,817,807, each incorporated herein by reference in their entirety).

Also incorporated by reference in its entirety are PCT publications WO 00/02870 and WO 01/44229, which describe additional compounds and also describe the structural characteristics of the cyclic polyamine antiviral agents. The structural characteristics of a number of non-cyclic amine antiviral agents have also been described in WO 00/56729, WO 02/22600, WO 02/22599, and WO 02/34745, each incorporated herein by reference in their entirety.

Improved methods for preparation of some of the cyclic polyamine compounds have also been described in U.S. Pat. Nos. 5,612,478; 5,756,728; 5,801,281; and 5,606,053 and PCT publication WO 02/26721, each of which is incorporated by reference herein in their entirety.

PCT publication WO 02/58653 teaches that some of the polyamine antiviral agents described in the above mentioned publications have the effect of increasing the white blood cell count. WO 03/011277 and U.S. patent publication US 2005/0043367 teach that some polyamine antiviral agents described in the above-mentioned publications also have the effect of increasing progenitor cells and/or stem cells.

The development and maturation of blood cells is a complex process. Mature blood cells are derived from hematopoietic precursor cells (progenitor) cells and stem cells present in specific hematopoietic tissues including bone marrow. Within these environments hematopoietic cells proliferate and differentiate prior to entering the circulation. The chemokine receptor CXCR4 and its natural ligand stromal cell derived factor-1 (SDF-1) appear to be important in this process (for reviews see Maekawa, T., et al., Internal Med. (2000) 39:90-100; Nagasawa, T., et al., Int. J. Hematol. (2000) 72:408-411). This is demonstrated by reports that CXCR4 or SDF-1 knock-out mice exhibit hematopoietic defects (Ma, Q., et al., Proc. Natl. Acad. Sci USA (1998) 95:9448-9453; Tachibana, K., et al., Nature (1998) 393:591-594; Zou, Y-R., et al., Nature (1998) 393:595-599). It is also known that CD34+ progenitor cells express CXCR4 and require SDF-1 produced by bone marrow stromal cells for chemoattraction and engraftment (Peled, A., et al., Science (1999) 283:845-848) and that in vitro, SDF-1 is chemotactic for both CD34+ cells (Aiuti, A., et al., J. Exp. Med. (1997) 185:111-120; Viardot, A., et al., Ann. Hematol. (1998) 77:194-197) and for progenitor/stem cells (Jo, D-Y., et al., J. Clin. Invest. (2000) 105:101-111). SDF-1 is also an important chemoattractant, signaling via the CXCR4 receptor, for several other more committed progenitors and mature blood cells including T-lymphocytes and monocytes (Bleul, C., et al., J. Exp. Med. (1996) 184:1101-1109), pro- and pre-B lymphocytes (Fedyk, E. R., et al., J. Leukoc. Biol. (1999) 66:667-673; Ma, Q., et al., Immunity (1999) 10:463-471) and megakaryocytes (Hodohara, K., et al., Blood (2000) 95:769-775; Riviere, C., et al., Blood (1999) 95:1511-1523; Majka, M., et al., Blood (2000) 96:4142-4151; Gear, A., et al., Blood (2001) 97:937-945; Abi-Younes, S., et al., Circ. Res. (2000) 86:131-138).

Thus, in summary, it appears that SDF-1 is able to control the positioning and differentiation of cells bearing CXCR4 receptors whether these cells are stem cells (i.e., cells which are CD34+) and/or progenitor cells (which result in formation of specified types of colonies in response to particular stimuli; that can be CD34⁺ or CD34⁻) or cells that are somewhat more differentiated.

Considerable attention has been focused on the number of CD34+ cells mobilized in the pool of peripheral blood progenitor cells used for autologous stem cell transplantation. The CD34+ population is the component thought to be primarily responsible for the improved recovery time after chemotherapy and the cells most likely responsible for long-term engraftment and restoration of hematopoiesis (Croop, J. M., et al., Bone Marrow Transplantation (2000) 26:1271-1279). The mechanism by which CD34+ cells re-engraft may be due to the chemotactic effects of SDF-1 on CXCR4 expressing cells (Voermans, C. Blood, 2001, 97, 799-804; Ponomaryov, T., et al., J. Clin. Invest. (2000) 106:1331-1339). More recently, adult hematopoietic stem cells were shown to be capable of restoring damaged cardiac tissue in mice (Jackson, K., et al., J. Clin. Invest. (2001) 107:1395-1402; Kocher, A., et al., Nature Med. (2001) 7:430-436).

There is growing evidence to show support for the concept of stem cell plasticity and the use of progenitor and/or stem cells, particularly stem cells in tissue repair (Orlic et al., Circ. Res. (2002 91:1092-1102)). Thus, the role of the CXCR4 receptor in managing cell positioning and differentiation has assumed considerable significance.

Citation of the above documents is not intended as an admission that any of the foregoing is pertinent prior art. All statements as to the date or representation as to the contents of these documents is based on the information available to the applicants and does not constitute any admission as to the correctness of the dates or contents of these documents. Further, all documents referred to throughout this application are incorporated in their entirety by reference herein.

### DISCLOSURE OF THE INVENTION

The invention is directed to methods of increasing blood flow and/or promoting tissue regeneration in a subject, particularly veterinary and human subjects, comprising directly administering mobilized and harvested progenitor and/or stem cells to target tissues in a subject. Compounds for use in the methods of the present invention include but are not limited to compounds described in the patents and publications incorporated herein by reference.

In one aspect, the invention provides methods for regenerating tissue in a subject, comprising directly administering mobilized and harvested progenitor and/or stem cells to damaged tissues in said subject, wherein the progenitor and/or stem cells are mobilized by a compound having formula (1), (2) or (3). The methods of present invention also provide methods for increasing blood flow to damaged tissues and/or for repairing damaged tissue.

The compounds of formula (1) are of the formula:

Z-linker-Z′ (1)

wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;

or Z is of the formula

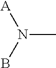

wherein A comprises a monocyclic or bicyclic fused ring system containing at least one N and B is H or an organic moiety of 1-20 atoms;

Z′ may be embodied in a form as defined by Z above, or alternatively may be of the formula

—N(R)—(CR₂)ₙ—X

wherein each R is independently H or straight, branched or cyclic alkyl (1-6C), n is 1 or 2, and

X is an aromatic ring, including heteroaromatic rings, or is a mercaptan;

or Z¹ is of the formula —Ar(Y)ⱼ;

wherein Ar is an aromatic or heteroaromatic moiety, and each Y is independently a non-interfering substituent and j is 0-3;

“linker” represents a bond, alkylene (1-6C) or may comprise aryl, fused aryl, oxygen atoms contained in an alkylene chain, or may contain keto groups or nitrogen or sulfur atoms.

The compounds of formula (2) are of the formula:

Z-linker-Z′ (2)

wherein Z is of the formula

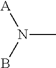

wherein A comprises a monocyclic or bicyclic fused ring system containing at least one N and B is H or an organic moiety of 1-20 atoms,

Z′ is of the formula

—Ar(Y)ⱼ;

wherein Ar is an aromatic or heteroaromatic moiety, and each Y is independently a non-interfering substituent and j is 0-3; and

“linker” represents a bond, alkylene (1-6C) or may comprise aryl, fused aryl, oxygen atoms contained in an alkylene chain, or may contain keto groups or nitrogen or sulfur atoms;

The compounds of formula (3) are of the formula:

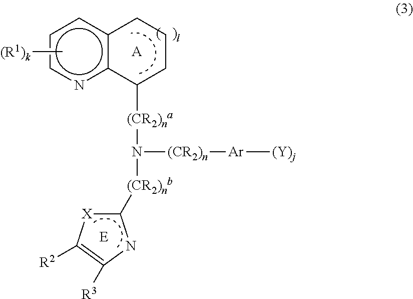

wherein:

Ring A optionally comprises a heteroatom selected from N, O and S;

the dotted lines represent optional unsaturation;

R1, R2 and R3 are non-interfering substituents;

k is 0-4;

l is 0, 1, or 2;

X is unsubstituted or substituted C or N; or is O or S;

Ar is the residue of an aromatic or heteroaromatic moiety;

each n is independently 0-2;

each R is independently H or alkyl (1-6C);

j is 0-3; and

each Y is independently halo, OH, SH, SO, SO₂, or an organic moiety of 1-20C atoms that does not contain N wherein two such Y may be connected to form a fused ring with Ar, or is selected from the group consisting of

—(CR₂)ₘCN,

—(CR₂)ₘNR⁵₂,

—(CR₂)ₘNR(CR₂)ₘNRR₄,

—(CR₂)ₘNR(CR₂)ₘNR(CR₂)ₘNR⁵₂,

—(CR₂)ₘCO(CR₂)ₘNR⁵₂,

—(CR₂)ₘCO(CR₂)ₘNR(CR₂)ₘNRR⁴,

—(CR₂)ₘCO(CR₂)NR(CR₂)ₘNR(CR₂)ₘNR⁵₂,

—(CR₂)ₘNRCO(CR₂)ₘNRR⁴,

—(CR₂)ₘNRCO(CR₂)ₘNR(CR₂)ₘNR⁵₂,

—(CR₂)ₘNRCO(CR₂)ₘNR(CR₂)ₘNR(CR₂)ₘNR(CR₂)ₘNR⁵₂,

—CH═N—Z″,

—(CR₂)ₘZ″,

NR(CR₂)ₘZ″,

—(CR₂)ₘNROH,

(CR₂)ₘCONROH, and

(CR₂)ₘCR═NOH,

wherein Z″ is an optionally substituted aromatic or heteroaromatic moiety containing 5-12 ring members; and

wherein R is as defined above, each m is independently 0-4, and R⁴ and each R⁵ is independently H, alkyl (1-6C), alkenyl (1-6C), alkynyl (1-6C), or acyl (1-6C), each optionally substituted by one or more nonaromatic, nonheterocyclic substituent(s), and wherein two R⁵ may be connected to form a cyclic amine, optionally containing one or more additional heteroatoms selected from N, O, and S.

In the compound of formula (3), ring E may be coupled to the remainder of the molecule at position 2. In one embodiment, R² and R³ taken together form a benzo substituent. In another embodiment, X is N and ring E comprises a pi bond coupled to one N. In yet another embodiment, k is 0-1. In one example, at least one Y is —CH₂NH₂. In other examples, Ar is the residue of benzene, benzimidazole, benzothiazole, imidazole, oxazole, benztriazole, thiazole, pyridine, or pyrimidine.

In the compound of formula (3), ring A may be saturated and 1 is 1. In one embodiment, the ring system which includes A is tetrahydroquinoline or a substituted form thereof. In other embodiments, one of (CR₂)^{a}ₙ and (CR₂)^{b}ₙ may be CH₂ and the other is a bond. In particular examples, (CR₂)^{a}ₙ is a bond and (CR₂)^{b}ₙ is CH₂.

In one embodiment, the progenitor and/or stem cells in the methods of the invention are mobilized and harvested by apheresis. In other embodiments, the progenitor and/or stem cells are mobilized into the peripheral blood or bone marrow of said subject and harvested. In some examples, the peripheral blood or bone marrow is derived from a subject who has been treated with granulocyte colony-stimulating factor. In other examples, the progenitor and/or stem cells are harvested from bone marrow.

In another embodiment, the damaged tissue that is regenerated using the methods of the present invention is ischemic tissue. The ischemic tissue may be an organ tissue or a limb tissue. Examples of organ tissue include but are not limited to myocardium, brain tissue, lung tissue, or liver tissue.

In particular examples, the progenitor and/or stem cells are mobilized by 1,1′-1,4-phenylene-bis-(methylene)-bis-1,4,8,11-tetraazacyclotetradecane (AMD 3100) or a pharmaceutically acceptable salt thereof. For example, the progenitor and/or stem cells may be mobilized by an acid addition salt of AMD 3100, such as AMD 3100 hydrochloride.

In the methods of the invention, the compound of formula (1) may be directly administered to damaged tissues in said subject in a dosage range of about 0.1 μg/kg-5 mg/kg of body weight. For example, the compound of formula (1) may be directly administered in the dosage range of about 1 μg/kg to 300 μg/kg of body weight, or in the dosage range of about 10 μg/kg to 100 μg/kg of body weight.

The methods of the present invention may be administered to a diabetic subject, or to a human subject that is suffering or has suffered from limb ischemia or a peripheral vascular disease, such as a peripheral arterial disease, or peripheral venous disorders. Examples of peripheral arterial disease include but are not limited to atherosclerosis, carotid artery disease, peripheral arterial disease of the legs, peripheral arterial disease of the renal arteries, abdominal aortic aneurysm, Raynaud syndrome, Buerger disease, or vasculitis.

In another aspect, the present invention provides methods for increasing blood flow in a subject, comprising directly administering mobilized and harvested progenitor and/or stem cells to a target tissue in said subject that is in need of increased blood flow, wherein the progenitor and/or stem are mobilized by a compound having formula (1), (2) or (3) as described above.

Furthermore, the present invention provides for the use of a compound having formula (1), (2) or (3) for the manufacture of a medicament for tissue regeneration, wherein said compound of formula (1), (2) or (3) is capable of mobilizing progenitor and/or stem cells, which are harvested and directly administered to damaged tissues in a subject. The present invention also provides for the use of a compound having formula (1), (2) or (3) for the manufacture of a medicament for increasing blood flow in a target tissue, wherein said compound having formula (1), (2) or (3) is capable of mobilizing progenitor and/or stem cells, which are harvested and directly administered to target tissues in a subject.

The methods of the invention also include treatment of cell populations ex vivo with the compounds of formula (1), (2) or (3), and directly administering the treated populations into damaged tissues in a compatible subject. The compounds of formula (1), (2) or (3) may be used alone or in combination with other compounds and compositions to enhance the population of stem cells and/or progenitor cells in the peripheral blood, thereby regenerating the damaged tissue. An enhanced production of white blood cells in the bone marrow may result as well.

### BRIEF DESCRIPTION OF THE DRAWINGS

FIG. 1 shows a graph of obtaining myeloid progenitors in response to treatment with 1,1′-[1,4-phenylene-bis(methylene)-bis-1,4,8,11-tetraazacyclotetradecane (AMD 3100) in combination with macrophage inflammatory protein after administration of granulocyte colony-stimulating factor (G-CSF).

FIG. 2 shows the percent of blood flow restoration in ischemic limbs followed by A) systemic administration of AMD 3100 in mice; and B) local injection of harvested CD34+ cells mobilized by AMD 3100.

### MODES OF CARRYING OUT THE INVENTION

The compounds useful in the invention are of the general formula set forth as formula (1)-(3) above. Certain embodiments are preferred; included among these are the compounds set forth in the above-incorporated U.S. patents and other patent documents. In particular examples, the compounds for use in the methods of the invention comprise AMD 3100.

In one embodiment, the compounds useful in the methods of the invention comprise cyclic polyamine and non-cyclic amine antiviral agents. As described in the above-mentioned documents, cyclic polyamine and non-cyclic amine antiviral agents inhibit HIV replication via inhibition of CXCR4, the co-receptor required for fusion and entry of T-tropic HIV strains, and also inhibit the binding and signaling induced by the natural ligand, the chemokine SDF-1. While not wishing to be bound by any theory, the compounds of formula (1)-(3) which inhibit the binding of SDF-1 to CXCR4 effect an increase in stem and/or progenitor cells by virtue of such inhibition. Enhancing the stem and/or progenitor cells in blood is helpful in treatments to alleviate the effects of protocols that adversely affect the bone marrow, such as those that result in leukopenia. These are known side-effects of chemotherapy and radiotherapy.

The compounds useful for the methods of the invention also enhance the success of bone marrow transplantation, enhance wound healing and burn treatment, and aid in restoration of damaged organ tissue. They also combat bacterial infections that are prevalent in leukemia. The compounds useful for the methods of the invention are used to mobilize and harvest CD34+ cells via apheresis with and without combinations with other mobilizing factors. The harvested cells are used in treatments requiring stem cell transplantations.

In one embodiment, progenitor and/or stem cells are mobilized by polyamine compounds, harvested and directly administered to target tissues in a subject that is in need of increased blood flow, particularly damaged tissues. For example, the harvested mobilized cells may be directly injected into cardiac tissue, neural tissue, ischemic tissue, or post-ischemic tissue. In other embodiments, mobilized and harvested progenitor and/or stem cells are systematically administered to the subject (e.g., subcutaneously or intraperitoneal administration). In yet other embodiments, mobilized and harvested progenitor and/or stem cells are administered directly to the heart muscle, left ventricle, right ventricle, coronary artery, peripheral circulation, or cerebro-spinal fluid.

In another aspect, the progenitor and/or stem cells may be further separated by selective purification to isolate specific cell populations. The homogenous extracted cells may than be administered as described in this invention.

As used herein, the term “progenitor cells” refers to cells that, in response to certain stimuli, can form differentiated hematopoietic or myeloid cells. The presence of progenitor cells can be assessed by the ability of the cells in a sample to form colony-forming units of various types, including, for example, CFU-GM (colony-forming units, granulocyte-macrophage); CFU-GEMM (colony-forming units, multipotential); BFU-E (burst-forming units, erythroid); HPP-CFC (high proliferative potential colony-forming cells); or other types of differentiated colonies which can be obtained in culture using known protocols.

As used herein, “stem” cells are less differentiated forms of progenitor cells. Typically, such cells are often positive for CD34. Some stem cells do not contain this marker, however. These CD34+ cells can be assayed using fluorescence activated cell sorting (FACS) and thus their presence can be assessed in a sample using this technique.

As used herein, “tissue” cells include but are not limited to cardiac tissue, brain tissue, peripheral vascular tissue, hepatic tissue, renal tissue, gastrointestinal tissue, lung tissue, liver tissue, smooth muscle tissue, or striated muscle tissue. As used herein, “damaged tissues” encompass any tissue in need of tissue regeneration, including but not limited to damaged tissues in need of increased blood flow. Tissues may be damaged as a result of a disease, such as heart disease and stroke.

As used herein, “peripheral vascular disease” refers to damage or dysfunction within peripheral arteries and veins.

In general, CD34+ cells are present only in low levels in the blood, but are present in large numbers in bone marrow. While other types of cells such as endothelial cells and mast cells also may exhibit this marker, CD34 is considered an index of stem cell presence.

In general, in compounds of formula (1), preferred embodiments of Z and Z′ are cyclic polyamine moieties having from 9-24C that include 3-5 nitrogen atoms. Particularly preferred are 1,5,9,13-tetraazacyclohexadecane; 1,5,8,11,14-pentaazacyclohexadecane; 1,4,8,11-tetraazacylotetradecane; 1,5,9-triazacyclododecane; 1,4,7,10-tetraazacyclododecane; and the like, including such cyclic polyamines which are fused to an additional aromatic or heteroaromatic rings and/or containing a heteroatom other than nitrogen incorporated in the ring. Embodiments wherein the cyclic polyamine contains a fused additional cyclic system or one or more additional heteroatoms are described in U.S. Pat. No. 5,698,546 and WO 01/44229 incorporated hereinabove by reference. Also preferred are
3,7,11,17-tetraazabicyclo(13.3.1)heptadeca-1(17),13,15-triene;
4,7,10,17-tetraazabicyclo(13.3.1)heptadeca-1 (17),13,15-triene;
1,4,7,10-tetraazacyclotetradecane; 1,4,7-triazacyclotetradecane; and
4,7,10-triazabicyclo(13.3.1)heptadeca-1(17),13,15-triene.

When Z′ is other than a cyclic polyamine as defined in Z, its preferred embodiments are set forth in U.S. Pat. No. 5,817,807, also incorporated herein by reference.

Preferred forms where

Z is of the formula

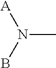

wherein A comprises a monocyclic or bicyclic fused ring system containing at least one N and B is H or an organic moiety of 1-20 atoms are disclosed in WO 00/56729; WO 02/22600; WO 02/34745; and WO 02/22599 cited above and all incorporated herein by reference.

In one embodiment, each “R” group in the compounds useful for the methods of the invention is independently straight or branched chain alkyl or may be cyclic, and may optionally be substituted by 1-2 substituents selected from halo, hydroxy and alkoxy. Preferably each R is H or lower straight-chain alkyl (1-4C), preferably methyl.

In the compounds of formula (1)-(3), Ar may be the residue of an aromatic or heteroaromatic moiety which contains a single or fused ring system and containing 5-6 ring members in the monocyclic system and 9-12 members in the fused ring system. The residue may be optionally substituted. Examples of optionally substituted aromatic and heteroaromatic groups include benzene, naphthalene, dihydronaphthalene, tetrahydronaphthalene, pyridine, quinoline, isoquinoline, imidazole, benzimidazole, azabenzimidazole, benzotriazole, furan, benzofuran, thiazole, benzothiazole, oxazole, benzoxazole, pyrrole, indole, imidazole, tetrahydroquinoline, tetrahydroisoquinoline, pyrazole, thiophene, isoxazole, isothiazole, triazole, tetrazole, oxadiazole, thiadiazole, imidazoline, and benzopyran. Oxides of the nitrogen and sulfur containing heteroaromatic rings are also included in the present invention. Particularly preferred forms of Ar are phenylene, pyridylene or pyridinylene.

When compounds of formula (1)-(3) contain elements that are “optionally substituted” these substituents are preferably halogen, nitro, cyano, carboxylic acid, optionally substituted alkyl, alkenyl or cycloalkyl groups, an optionally substituted hydroxyl group, an optionally substituted thiol group, an optionally substituted amino, an optionally substitute acyl group, an optionally substituted carboxylate, carbamate, carboxamide or sulfonamide group, or an optionally substituted aromatic or heterocyclic group.

Examples of halogen include fluorine, chlorine, bromine, iodine, etc., with fluorine and chlorine preferred.

Examples of optionally substituted alkyl include C₁₋₁₀ alkyl, including methyl, ethyl propyl, etc.; examples of optionally substituted alkenyl groups include C₂₋₁₀ alkenyl such as allyl, crotyl, 2-pentenyl, 3-hexenyl, etc.; and examples of optionally substituted cycloalkyl groups include C₃₋₁₀ cycloalkyl such as cyclopropyl, cyclobutyl, cyclopentyl, cyclohexyl, cycloheptyl, etc. In these cases, C₁₋₆ alkyl, alkenyl and cycloalkyl are preferred. The optional substituent may also be an optionally substituted aralkyl (e.g., phenyl C₁₋₄ alkyl) or heteroalkyl for example, phenylmethyl (benzyl), phenylethyl, pyridinylmethy, pyridinylethyl, etc. The heterocyclic group may be a 5 or 6 membered ring containing 1-4 heteroatoms.

Examples of optionally substituted hydroxyl and thiol groups include those wherein the substituent is an optionally substituted alkyl (e.g., C₁₋₁₀ alkyl) such as methyl, ethyl, propyl, isopropyl, butyl, isobutyl, sec-butyl, tert-butyl, pentyl, etc., preferably (C₁₋₆) alkyl; an optionally substituted cycloalkyl (e.g., C₃₋₇ cycloalkyl, etc., such as cyclopropyl, cyclobutyl, cyclopentyl, cyclohexyl, cycloheptyl, etc.); an optionally substituted aralkyl (e.g., phenyl-C₁₋₄ alkyl, e.g., benzyl, phenethyl, etc.). Where there are two adjacent hydroxyl or thiol substituents, the heteroatoms may be connected via an alkylene group such as O(CH₂)ₙO and S(CH₂)ₙS (where n=1-5). Examples include methylenedioxy, ethylenedioxy, etc. Oxides of thio-ether groups such as sulfoxides and sulfones are also encompassed.

Further examples of the optionally substituted hydroxyl group include an optionally substituted C₂₋₄ alkanoyl (e.g., acetyl, propionyl, butyryl, isobutyryl, etc.), C₁₋₄ alkylsulfonyl (e.g., methane sulfonyl, ethanesulfonyl, etc.) and an optionally substituted aromatic and heterocyclic carbonyl group including benzoyl, pyridinecarbonyl, etc.

The substituents on optionally substituted amino group may bind to each other to form a cyclic amino group (e.g., 5- to 6-membered cyclic amino, etc., such as tetrahydropyrrole, piperazine, piperidine, pyrrolidine, morpholine, thiomorpholine, pyrrole, imidazole, etc.). Said cyclic amino group may have a substituent, and examples of the substituents include halogen (e.g., fluorine, chlorine, bromine, iodine, etc.), nitro, cyano, hydroxy group, thiol group, amino group, carboxyl group, an optionally halogenated C₁₋₄ alkyl (e.g., trifluoromethyl, methyl, ethyl, etc.), an optionally halogenated C₁₋₄ alkoxy (e.g., methoxy, ethoxy, trifluoromethoxy, trifluoroethoxy, etc.), C₂₋₄ alkanoyl (e.g., acetyl, propionyl, etc.), C₁₋₄ alkylsulfonyl (e.g., methanesulfonyl, ethanesulfonyl, etc.) the number of preferred substituents are 1 to 3.

The amino group may also be substituted once or twice (to form a secondary or tertiary amine) with a group such as an optionally substituted alkyl group including C₁₋₁₀ alkyl (e.g., methyl, ethyl propyl, etc.); an optionally substituted alkenyl group such as allyl, crotyl, 2-pentenyl, 3-hexenyl, etc., or an optionally substituted cycloalkyl group such as cyclopropyl, cyclobutyl, cyclopentyl, cyclohexyl, cycloheptyl, etc. In these cases, C₁₋₆ alkyl, alkenyl and cycloalkyl are preferred. The amine group may also be optionally substituted with an aromatic or heterocyclic group, aralkyl (e.g., phenyl C₁₋₄ alkyl) or heteroalkyl for example, phenyl, pyridine, phenylmethyl (benzyl), phenethyl, pyridinylmethyl, pyridinylethyl, etc. The heterocyclic group may be a 5 or 6 membered ring containing 1-4 heteroatoms. The optional substituents of the “optionally substituted amino groups are the same as defined above for the “optionally substituted cyclic amino group.”

The amino group may be substituted with an optionally substituted C₂₋₄ alkanoyl, e.g., acetyl, propionyl, butyryl, isobutyryl etc., or a C₁₋₄ alkylsulfonyl (e.g., methanesulfonyl, ethanesulfonyl, etc.) or a carbonyl or sulfonyl substituted aromatic or heterocyclic ring, e.g., benzenesulfonyl, benzoyl, pyridinesulfonyl, pyridinecarbonyl, etc. The heterocycles are as defined above.

Examples of the optionally substituted acyl groups include a carbonyl group or a sulfinyl or sulfonyl group binding to hydrogen; or to an optionally substituted alkyl (e.g., C₁₋₁₀ alkyl such as methyl, ethyl, propyl, isopropyl, butyl, isobutyl, sec-butyl, tert-butyl, pentyl, isopentyl, neopentyl, hexyl, heptyl, octyl, nonyl, decyl, etc., preferably lower (C₁₋₆) alkyl, etc.; an optionally substituted cycloalkyl (e.g., C₃₋₇ cycloalkyl, etc., such as cyclopropyl, cyclobutyl, cyclopentyl, cyclohexyl, cycloheptyl, etc.); an optionally substituted alkenyl (e.g., C₂₋₁₀ alkenyl such as allyl, crotyl, 2-pentenyl, etc., preferably lower (C₂₋₆) alkenyl, etc.); an optionally substituted cycloalkenyl (e.g., C₃₋₇ cycloalkenyl, etc., such as 2-cyclopentenyl, 2-cyclohexenyl, 2-cyclopentenylmethyl, 2-cyclohexenylmethyl, etc.) an optionally substituted 5- to 6-membered monocyclic aromatic group (e.g., phenyl, pyridyl, etc.).

Examples of the optionally substituted carboxylate group (ester groups) include an optionally substituted alkyl (e.g., C₁₋₁₀ alkyl such as methyl, ethyl, propyl, isopropyl, butyl, isobutyl, sec-butyl, tert-butyl, pentyl, isopentyl, neopentyl, hexyl, heptyl, octyl, nonyl, decyl, etc., preferably lower (C₁₋₆) alkyl, etc.); an optionally substituted cycloalkyl, e.g., C₃₋₇ cycloalkyl, etc. such as cyclopropyl, cyclobutyl, cyclopentyl, cyclohexyl, cycloheptyl, etc.); an optionally substituted alkenyl (e.g., C₂₋₁₀ alkenyl such as allyl, crotyl, 2-pentenyl, 3-hexenyl, etc., preferably lower (C₂₋₆) alkenyl, etc.); an optionally substituted cycloalkenyl e.g., C₃₋₇ cycloalkenyl, etc., such as 2-cyclohexenylmethyl, etc.); an optionally substituted aryl e.g., phenyl, naphthyl, etc.) and C₁₋₄ aryl for example, benzyl, phenethyl etc. Groups such as methoxymethyl, methoxyethyl, etc., are also encompassed.

Examples of the optionally substituted carboxamide and sulfonamide groups are identical in terms of the amine definition as the “optionally substituted amino group” defined above.

Examples of the optionally substituted aromatic or heterocyclic groups are phenyl, naphthyl, or a 5- or 6-membered heterocyclic ring containing 1-4 heteroatoms. The optional substituents are essentially identical to those listed above.

The non-interfering substituents R¹, R² and R³ in formula (1)-(3) are similar to those set forth as “optional substituents”. Preferably, R¹ is selected from the optional substituents set forth above, preferably halo, substituted or unsubstituted alkyl, substituted or unsubstituted hydroxyl, substituted or unsubstituted amino, substituted or unsubstituted thiol, and substituted or unsubstituted acyl. Preferably k is 0-2, preferably 0-1, and more preferably 0.

The substituents R² and R³ are preferably selected from the preferred embodiments of R¹ listed immediately above, or, more preferably, may be joined to form a saturated or unsaturated ring system, preferably a benzo ring system.

In the above formula (3), examples of the optionally substituted ring system containing ring A are dihydroquinoline, tetrahydroquinoline, pyranopyridine, dihydropyranopyridine, thiapyranopyridine, dihydrothiapyranopyridine, dihydronaphthyridine, tetrahydronaphthyridine. Oxides of sulfur-containing heterocycles are also encompassed in the present invention. In the above ring system containing Ring A, the optional nitrogen atom may be substituted with hydrogen, a substituted alkyl, alkenyl, cycloalkyl or aryl group, or may be the nitrogen atom of a carboxamide, carbamate or sulfonamide. Preferred for 1 is l=1, it is preferred that ring A be saturated. The most preferred combination is tetrahydroquinoline.

In the above formula (3), X may be CH (pyrrole), O (oxazole), S (thiazole), NH or NR (imidazole) where R is a C₁₋₆ alkyl group or acyl, sulfonyl group. In Formula 1, two adjacent R¹ and/or R² and R³ may be joined to form an optionally substituted, fused 5-7 membered ring. Examples of fused ring systems include but are not limited to indole, tetrahydroindole, benzimidazole, tetrahydrobenzimidazole, azabenzimidazole, benzoxazole, tetrahydrobenzoxazole, benzothiazole, tetrahydrobenzothiazole. The preferred ring systems resulting from R² and R³ include those which result in benzothiazole and benzoimidazole.

In the compounds of formula (3), it is preferred that one of the (CR₂)ₙ linkers between the ring system containing ring A and ring E is that wherein n is 0, i.e., the linkage is merely a covalent bond. Also preferred embodiments of (CR₂)ₙ in this context are ethylene or methylene, preferably methylene. In the most preferred embodiments, the linkage between the nitrogen shown in formula 2 and ring A is a bond and that between the nitrogen shown and ring E is CH₂. As shown, ring E may be coupled to the linker through any position, but preferably through position 2, 4 or 5, most preferably through position 2.

In the compounds of formula (3), preferred values of j are 0-2, preferably 1-2. The embodiments of Y may be varied widely provided Y does not contain nitrogen. Thus, Y may be halo, OH, SH, SO, SO₂ and the like, or a substituent of 1-20 carbons, optionally containing as a substitution, for one or more said carbons, a heteroatom such as O or S. Preferred embodiments wherein N is not present in Y include halo, optionally substituted alkyl, optionally substituted hydroxyl, optionally substituted thiol, and optionally substituted carboxylate, and a saturated or unsaturated ring. These substituents are described above. Where N is included in Y, Y is selected from the moieties set forth hereinabove. In these substituents, Z″ is an aromatic or heteroaromatic moiety containing 5-12 ring members. Thus, Y may include a single or fused ring. Examples of preferred forms of Z″ are identical to those set forth with regard to the aromatic residue Ar set forth above, but are monovalent.

As shown, in certain embodiments, R, defined as H or alkyl (1-6C), is replaced by R⁴ or R⁵ which have a broader definitions and can include the embodiments of R as well as embodying optionally substituted alkenyl, acyl, and the like as set forth above. Preferred forms of R⁴ and R⁵ include those typified by R and optionally substituted alkenyl. Also preferred are embodiments where two R⁵ are connected to form a cyclic amine, including those which contain one or more additional heteroatoms such as N, O, and/or S.

Preferred forms of Y when Y contains N are those wherein R is in all cases H or methyl, preferably H and those where two R⁵ are coupled. Especially preferred are those of the formula

—(CR₂)ₘCN,

—(CR₂)ₘNR⁵₂,

—(CR₂)ₘNR(CR₂)ₘNRR⁴,

—(CR₂)ₘCO(CR₂)ₘNR⁵₂,

—(CR₂)ₘZ″, and

—NR(CR₂)ₘZ″,

and those wherein Y comprises guanidino or NHNHR,

especially wherein (CR₂)ₘ is CH₂, CH₂CH₂, or CH₂CH₂CH₂, or wherein m is 0, and those wherein R⁴ or R⁵ is H or is lower alkyl, alkenyl, or hydrogen, or wherein both R⁵ are identical.

Particularly preferred are —CH₂NH₂, CH₂CH₂NH₂, —CH₂NMe₂, —CH₂CH₂NMe₂, —CONH₂, —CONMe₂, and the like.

Preferred Z″ are optionally substituted residues of benzene, oxazole, imidazole, thiazole, benzimidazole, benzthiazole, benzoxazole, indole, thiophene, tetrazine, pyrimidine, pyridine, and the like.

Preferred forms of the linker moiety include those wherein the linker is a bond, or wherein the linker includes an aromatic moiety flanked by alkylene, preferably methylene moieties. Preferred linking groups include the methylene bracketed forms of 1,3-phenylene, 2,6-pyridine, 3,5-pyridine, 2,5-thiophene, 4,4′-(2,2′-bipyrimidine); 2,9-(1,10-phenanthroline) and the like. A particularly preferred linker is 1,4-phenylene-bis-(methylene).

Particularly preferred embodiments of the compound of the formula (1) include 2,2′-bicyclam; 6,6′-bicyclam; the embodiments set forth in U.S. Pat. Nos. 5,021,409, and 6,001,826, and in particular 1,1′-[1,4-phenylene-bis(methylene)]-bis-1,4,8,11-tetraazacyclotetradecane, set forth in U.S. Pat. No. 5,583,131, and designated herein AMD3100.

Other preferred embodiments include
N-[1,4,8,11-tetraazacyclotetradecanyl-1,4-phenylenebis(methylene)]-2-aminomethyl)pyridine;
7,7′-[1,4-phenylenebis(methylene)]bis-4,7,10,17-tetraazabicyclo-[13.3.1]heptadeca-1(17),13,15-triene;
7,7′-[1,4-phenylenebis(methylene)]bis-3,7,11,17-tetraazabicyclo[13.3.1]heptadeca-1(17),13,15-triene;
1,1′-[1,3-phenylenebis(methylene)]-bis-1,4,8,11-tetra-azacyclotetradecane;
1,1′-[1,4-phenylenebis(methylene)]-bis-1,4,8,11-tetra-azacyclotetradecane;
1,1′-[1,4-phenylene-bis-(methylene)]-bis-1,4,7,10-tetraazacyclotetradecane;
1,1′-[1,3-phenylene-bis-(methylene)]-bis-1,4,7,10-tetraazacyclotetradecane;
11,11′-(1,2-propanediyl)bis-1,4,8,11-tetraazacyclotetradecane;
N-[4-(1,4,7-triazacyclotetra-decane)-1,4-phenylenebis(methylene)]-2-(aminomethyl)pyridine;
N-[7-(4,7,10-triazabicyclo[13.3.1]heptadeca-1 (17),13,15-triene)-1,4-phenylenebis(methylene)]-2-(aminomethyl)pyridine;
N-[7-(4,7,10,17-tetraazabicyclo[13.3.1]heptadeca-1 (17),13,15-triene)-1,4-phenylenebis(methylene)]-2-(aminomethyl)pyridine;
N-[4-[4,7,10,17-tetraazabicyclo[13.3.1]heptadeca-1 (17),13,15-triene]-1,4-phenylenebis(methylene)]-2-(aminomethyl)pyridine;
3,3′-(bis-1,5,9,13-tetraazacyclohexadecane);
3,3′-(bis-1,5,8,11,14-pentaazacyclohexadecane), methylene (or polymethylene) di-1-N-1,4,8,11-tetraazacyclotetradecane;
3,3′-bis-1,5,9,13,-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
11,11′(1,2-ethanediyl)bis-1,4,8,11-tetraazacyclotetradecane;
11,11′-(1,2-propanediyl)bis-1,4,8,11-tetraazacyclotetradecane;
11,11′-(1,2-butanediyl)bis-1,4,8,11-tetraazacyclotetradecane;
11,11′-(1,2-pentanediyl)bis-1,4,8,11-tetraazacyclotetradecane;
11,11′-(1,2-hexanediyl)bis-1,4,8,11-tetraazacyclotetradecane;
3,3′-bis-1,5,9,13-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
11,11′-(1,2-ethanediyl)bis-1,4,8,11-tetraazacyclotetradecane;
11,11′-(1,2-propanediyl)bis-1,4,8,11-tetraazacyclotetradecane;
11,11′-(1,2-butanediyl)bis-1,4,8,11-tetraazacyclotetradecane;
11,11′-(1,2-pentanediyl)bis-1,4,8,11-tetraazacyclotetradecane;
11,11′-(1,2-hexanediyl)bis-1,4,8,11-tetraazacyclotetradecane;
1,1′-[1,3-phenylenebis(methylene)]-bis-1,4,8,11-tetra-azacyclotetradecane;
1,1′-[1,4-phenylenebis(methylene)]-bis-1,4,8,11-tetra-azacyclotetradecane;
1,1′-[3,3′-biphenylene-bis-(methylene)]-bis-1,4,8,11-tetraazacyclotetradecane;
11,11′-[1,4-phenylene-bis-(methyl ene)]-bis-1,4,7,11-tetraazacyclotetradecane;
1,11′-[1,4-phenylene-bis(methylene)]-1,4,8,11-tetraazacyclotetradecane;
1,1′-[2,6-pyridine-bis-(methylene)]-bis-1,4,8,11-tetraazacyclotetradecane;
1,1′-[3,5-pyridine-bis-(methylene)]-bis-1,4,8,11-tetraazacyclotetradecane;
1,1′-[2,5-thiophene-bis-(methylene)]-bis-1,4,8,11-tetraazacyclotetradecane;
1,1′-[4,4′-(2,2′-bipyridine)-bis-(methylene)]-bis-1,4,8,11-tetraazacyclotetradecane;
1,1′-[2,9-(1,10-phenanthroline)-bis-(methylene)]bis-1,4,8,11-tetraazacyclotetradecane;
1,1′-[1,3-phenylene-bis-(methylene)]-bis-1,4,7,10-tetraazacyclotetradecane;
1,1′-[1,4-phenylene-bis-(methylene)]-bis-1,4,7,10-tetraazacyclotetradecane;
1,1′-[5-nitro-1,3-phenylenebis(methylene)]bis-1,4,8,11-tetraazacyclotetradecane;
1,1′-[2,4,5,6-tetrachloro-1,3-phenyleneis(methylene)]bis-1,4,8,11-tetraazacyclotetradecane;
1,1′-[2,3,5,6-tetrafluoro-1,4-phenylenebis(methylene)]bis-1,4,8,11-tetraazacyclotetradecane;
1,1′-[1,4-naphthylene-bis-(methylene)]bis-1,4,8,11-tetraazacyclotetradecane;
1,1′-[1,3-phenylenebis-(methylene)]bis-1,5,9-triazacyclododecane;
1,1′-[1,4-phenylene-bis-(methylene)]-1,5,9-triazacyclododecane;
1,1′-[2,5-dimethyl-1,4-phenylenebis-(methylene)]-bis-1,4,8,11-tetraazacyclotetradecane;
1,1′-[2,5-dichloro-1,4-phenylenebis-(methylene)]-bis-1,4,8,11-tetraazacyclotetradecane;
1,1′-[2-bromo-1,4-phenylenebis-(methylene)]-bis-1,4,8,11-tetraazacyclotetradecane;
1,1′-[6-phenyl-2,4-pyridinebis-(methylene)]-bis-1,4,8,11-tetraazacyclotetradecane;
7,7′-[1,4-phenylene-bis(methylene)]bis-3,7,11,17-tetraazabicyclo[13.3.1]heptadeca-1(17),13,15-triene;
7,7′-[1,4-phenylene-bis(methylene)]bis[15-chloro-3,7,11,17-tetraazabicyclo[13.3.1]heptadeca-1(17),13,15-triene];
7,7′-[1,4-phenylene-bis(methylene)]bis[15-methoxy-3,7,11,17-tetraazabicyclo[13.3.1]heptadeca-1(17),13,15-triene];
7,7′-[1,4-phenylene-bis(methylene)]bis-3,7,11,17-tetraazabicyclo[13.3.1]-heptadeca-13,16-triene-15-one;
7,7′-[1,4-phenylene-bis(methylene)]bis-4,7,10,17-tetraazabicyclo[13.3.1]-heptadeca-1(17),13,15-triene;
8,8′-[1,4-phenylene-bis(methylene)]bis-4,8,12,19-tetraazabicyclo[15.3.1]nonadeca-1(19),15,17-triene;
6,6′-[1,4-phenylene-bis(methylene)]bis-3,6,9,15-tetraazabicyclo[11.3.1]pentadeca-1(15),11,13-triene;
6,6′-[1,3-phenylene-bis(methylene)]bis-3,6,9,15-tetraazabicyclo[11.3.1]pentadeca-1(15),11,13-triene;
17,17′-[1,4-phenylene-bis(methylene)]bis-3,6,14,17,23,24-hexaazatricyclo[17.3.1.1^{8,12}]tetracosa-1(23),8,10,12(24),19,21-hexaene;
N-[1,4,8,11-Tetraazacyclotetradecanyl-1,4-phenylenebis(methylene)]-2-(amino-methyl)pyridine;
N-[1,4,8,11-Tetraazacyclotetradecanyl-1,4-phenylenebis(methylene)]-N-methyl-2-(aminomethyl)pyridine;
N-[1,4,8,11-Tetraazacyclotetradecanyl-1,4-phenylenebis(methylene)]-4-(amino-methyl)pyridine;
N-[1,4,8,11-Tetraazacyclotetradecanyl-1,4-phenylenebis(methylene)]-3-(amino-methyl)pyridine;
N-[1,4,8,11-Tetraazacyclotetradecanyl-1,4-phenylenebis(methylene)]-(2-amino-methyl-5-methyl)pyrazine;
N-[1,4,8,11-Tetraazacyclotetradecanyl-1,4-phenylenebis(methylene)]-2-(amino-ethyl)pyridine;
N-[1,4,8,11-Tetraazacyclotetradecanyl-1,4-phenylenebis(methylene)]-2-(amino-methyl)thiophene;
N-[1,4,8,11-Tetraazacyclotetradecanyl-1,4-phenylenebis(methylene)]-2-(amino-ethyl)mercaptan;
N-[1,4,8,11-Tetraazacyclotetradecanyl-1,4-phenylenebis(methylene)]-2-amino-benzylamine;
N-[1,4,8,11-Tetraazacyclotetradecanyl-1,4-phenylenebis(methylene)]-4-amino-benzylamine;
N-[1,4,8,11-Tetraazacyclotetradecanyl-1,4-phenylenebis(methylene)]-4-(amino-ethyl)imidazole;
N-[1,4,8,11-Tetraazacyclotetradecanyl-1,4-phenylenebis(methylene)]-benzylamine;
N-[1,4,8,11-Tetraazacyclotetradecanyl-1,4-phenylenebis(methylene)]-purine;
N-[1,4,8,11-Tetraazacyclotetradecanyl-1,4-phenylenebis(methylene)]-4-phenylpiperazine;
N-[4-(1,4,7-Triazacyclotetra-decanyl)-1,4-phenylenebis(methylene)]-2-(aminomethyl)pyridine;
N-[7-(4,7,10,17-Tetraazabicyclo[13.3.1]heptadeca-1(17),13,15-trienyl)-1,4-phenylenebis(methylene)]-2-(aminomethyl)pyridine;
N-[7-(4,7,10-Triazabicyclo[13.3.1]heptadeca-1 (17),13,15-trienyl)-1,4-phenylenebis(methylene)]-2-(aminomethyl)pyridine;
N-[4-[4,7,10-Triazabicyclo[13.3.1]heptadeca-1 (17),13,15-trienyl]-1,4-phenylenebis(methylene)]-2-(aminomethyl)pyridine;
N-[1-(1,4,7-Triazacyclotetra-decanyl)-1,4-phenylenebis(methylene)]-2-(aminomethyl)pyridine;
N-[4-[4,7,10,17-Tetraazabicyclo[13.3.1]heptadeca-1(17),13,15-trienyl]-1,4-phenylenebis(methylene)]-2-(aminomethyl)pyridine;
N-[3-(3,6,17-Triazabicyclo[13.3.1]heptadeca-1 (17),13,15-trienyl)-1,4-phenylenebis(methylene)]-2-(aminomethyl)pyridine;
N-[3-(3,6,17-Triazabicyclo[13.3.1]heptadeca-1 (17),13,15-trienyl)-1,3-phenylenebis(methylene)]-2-(aminomethyl)pyridine;
N-[4-(4,7,17-Triazabicyclo[13.3.1]heptadeca-1(17),13,15-trienyl)-1,4-phenylenebis(methylene)]-2-(aminomethyl)pyridine;
N-[7-(4,7,17-Triazabicyclo[13.3.1]heptadeca-1 (17),13,15-trienyl)-1,4-phenylenebis(methylene)]-2-(aminomethyl)pyridine;
N-[6-(3,6,9-Triazabicyclo[11.3.1]pentadeca-1 (15),11,13-trienyl)-1,3-phenylenebis(methylene)]-2-(aminomethyl)pyridine;
N-[7-(4,10,17-Triazabicyclo[13.3.1]heptadeca-1(17),13,15-trienyl)-1,4-phenylenebis(methylene)]-2-(aminomethyl)pyridine;
N-[4-(1,7-Diazacyclotetradecanyl)-1,4-phenylenebis(methylene)]-2-(aminomethyl)pyridine;
N-[7-(4,10-Diazabicyclo[13.3.1]heptadeca-1 (17),13,15-trienyl)-1,4-phenylenebis(methylene)]-2-(aminomethyl)pyridine;
N-[4-(11-Fluoro-1,4,7-triazacyclotetradecanyl)-1,4-phenylenebis(methylene)]-2-(aminomethyl)pyridine;
N-[4-(11,11-difluoro-1,4,7-triazacyclotetradecanyl)-1,4-phenylenebis(methylene)]-2-(aminomethyl)pyridine;
N-[4-(1,4,7-triazacyclotetradecan-2-one)-yl))-1,4-phenylenebis(methylene)]-2-(aminomethyl)pyridine;
N-[12-(5-oxa-1,9-diazacyclotetradecanyl)-1,4-phenylenebis(methylene)]-2-(aminomethyl)pyridine;
N-[4-(11-oxa-1,7-diazacyclotetradecanyl)-1,4-phenylenebis(methylene)]-2-(aminomethyl)pyridine;
N-[4-(11-thia-1,7-diazacyclotetradecanyl)-1,4-phenylenebis(methylene)]-2-(aminomethyl)pyridine;
N-[4-(11-sulfoxo-1,7-diazacyclotetradecanyl)-1,4-phenylenebis(methylene)]-2-(aminomethyl)pyridine;
N-[4-(11-sulfono-1,7-diazacyclotetradecanyl)-1,4-phenylenebis(methylene)]-2-(aminomethyl)pyridine;
N-[4-(1,4,7-triazacyclotetradecan-3-one)-yl))-1,4-phenylenebis(methylene)]-2-(aminomethyl)pyridine;
N-(2-pyridinylmethyl)-N′-(6,7,8,9-tetrahydro-5H-cyclohepta[b]pyridin-9-yl)-1,4-benzenedimethanamine;
N-(2-pyridinylmethyl)-N′-(5,6,7,8-tetrahydro-8-quinolinyl)-1,4-benzenedimethanamine;
N-(2-pyridinylmethyl)-N′-(6,7-dihydro-5H-cyclopenta[b]pyridin-7-yl)-1,4-benzenedimethanamine;
N-(2-pyridinylmethyl)-N′-(1,2,3,4-tetrahydro-1-naphthalenyl)-1,4-benzenedimethanamine;
N-(2-pyridinylmethyl)-N′-(1-naphthalenyl)-1,4-benzenedimethanamine;
N-(2-pyridinylmethyl)-N′-(8-quinolinyl)-1,4-benzenedimethanamine;
N-(2-pyridinylmethyl)-N′-[2-[(2-pyridinylmethyl)amino]ethyl]-N′-(1-methyl-1,2,3,4-tetrahydro-8-quinolinyl)-1,4-benzene dimethanamine;
N-(2-pyridinylmethyl)-N′-[2-[(1H-imidazol-2-ylmethyl)amino]ethyl]-N′-(1-methyl-1,2,3,4-tetrahydro-8-quinolinyl)-1,4-benzene dimethanamine;
N-(2-pyridinylmethyl)-N′-(1,2,3,4-tetrahydro-8-quinolinyl)-1,4-benzenedimethanamine;
N-(2-pyridinylmethyl)-N′-[2-[(1H-imidazol-2-ylmethyl)amino]ethyl]-N′-(1,2,3,4-tetrahydro-1-naphthalenyl)-1,4-benzene dimethanamine;
N-(2-pyridinylmethyl)-N′-(2-phenyl-5,6,7,8-tetrahydro-8-quinolinyl)-1,4-benzenedimethanamine;
N,N′-bis(2-pyridinylmethyl)-N′-(2-phenyl-5,6,7,8-tetrahydro-8-quinolinyl)-1,4-benzenedimethanamine;
N-(2-pyridinylmethyl)-N′-(5,6,7,8-tetrahydro-5-quinolinyl)-1,4-benzenedimethanamine;
N-(2-pyridinylmethyl)-N′-(1H-imidazol-2-ylmethyl)-N′-(5,6,7,8-tetrahydro-5-quinolinyl)-1,4-benzenedimethanamine;
N-(2-pyridinylmethyl)-N′-(1H-imidazol-2-ylmethyl)-N′-(5,6,7,8-tetrahydro-8-quinolinyl)-1,4-benzenedimethanamine;
N-(2-pyridinylmethyl)-N′-[(2-amino-3-phenyl)propyl]-N′-(5,6,7,8-tetrahydro-8-quinolinyl)-1,4-benzenedimethanamine;
N-(2-pyridinylmethyl)-N′-(1H-imidazol-4-ylmethyl)-N′-(5,6,7,8-tetrahydro-8-quinolinyl)-1,4-benzenedimethanamine;
N-(2-pyridinylmethyl)-N′-(2-quinolinylmethyl)-N′-(5,6,7,8-tetrahydro-8-quinolinyl)-1,4-benzenedimethanamine;
N-(2-pyridinylmethyl)-N′-(2-(2-naphthoyl)aminoethyl)-N′-(5,6,7,8-tetrahydro-8-quinolinyl)-1,4-benzenedimethanamine;
N-(2-pyridinylmethyl)-N′-[(S)-(2-acetylamino-3-phenyl)propyl]-N′-(5,6,7,8-tetrahydro-8-quinolinyl)-1,4-benzenedimethanamine;
N-(2-pyridinylmethyl)-N′-[(S)-(2-acetylamino-3-phenyl)propyl]-N′-(5,6,7,8-tetrahydro-8-quinolinyl)-1,4-benzenedimethanamine;
N-(2-pyridinylmethyl)-N′-[3-((2-naphthalenylmethyl)amino)propyl]-N′-(5,6,7,8-tetrahydro-8-quinolinyl)-1,4-benzenedimethanamine;
N-(2-pyridinylmethyl)-N′-[2-(S)-pyrollidinylmethyl]-N′-(5,6,7,8-tetrahydro-8-quinolinyl)-1,4-benzenedimethanamine;
N-(2-pyridinylmethyl)-N′-[2-(R)-pyrollidinylmethyl]-N′-(5,6,7,8-tetrahydro-8-quinolinyl)-1,4-benzenedimethanamine;
N-(2-pyridinylmethyl)-N′-[3-pyrazolylmethyl]-N′-(5,6,7,8-tetrahydro-8-quinolinyl)-1,4-benzenedimethanamine;
N-(2-pyridinylmethyl)-N′-[2-pyrrolylmethyl]-N′-(5,6,7,8-tetrahydro-8-quinolinyl)-1,4-benzenedimethanamine;
N-(2-pyridinylmethyl)-N′-[2-thiopheneylmethyl]-N′-(5,6,7,8-tetrahydro-8-quinolinyl)-1,4-benzenedimethanamine
N-(2-pyridinylmethyl)-N′-[2-thiazolylmethyl]-N′-(5,6,7,8-tetrahydro-8-quinolinyl)-1,4-benzenedimethanamine;
N-(2-pyridinylmethyl)-N′-[2-furanylmethyl]-N′-(5,6,7,8-tetrahydro-8-quinolinyl)-1,4-benzenedimethanamine;
N-(2-pyridinylmethyl)-N′-[2-[(phenylmethyl)amino]ethyl]-N′-(5,6,7,8-tetrahydro-8-quinolinyl)-1,4-benzenedimethanamine;
N-(2-pyridinylmethyl)-N′-(2-aminoethyl)-N′-(5,6,7,8-tetrahydro-8-quinolinyl)-1,4-benzenedimethanamine;
N-(2-pyridinylmethyl)-N′-3-pyrrolidinyl-N′-(5,6,7,8-tetrahydro-8-quinolinyl)-1,4-benzenedimethanamine
N-(2-pyridinylmethyl)-N′-4-piperidinyl-N′-(5,6,7,8-tetrahydro-8-quinolinyl)-1,4-benzenedimethanamine;
N-(2-pyridinylmethyl)-N′-[2-[(phenyl)amino]ethyl]-N′-(5,6,7,8-tetrahydro-8-quinolinyl)-1,4-benzenedimethanamine;
N-(2-pyridinylmethyl)-N′-(7-methoxy-1,2,3,4-tetrahydro-2-naphthalenyl)-1,4-benzenedimethanamine;
N-(2-pyridinylmethyl)-N′-(6-methoxy-1,2,3,4-tetrahydro-2-naphthalenyl)-1,4-benzenedimethanamine;
N-(2-pyridinylmethyl)-N′-(1-methyl-1,2,3,4-tetrahydro-2-naphthalenyl)-1,4-benzenedimethanamine;
N-(2-pyridinylmethyl)-N′-(7-methoxy-3,4-dihydronaphthalenyl)-1-(aminomethyl)-4-benzamide;
N-(2-pyridinylmethyl)-N′-(6-methoxy-3,4-dihydronaphthalenyl)-1-(aminomethyl)-4-benzamide;
N-(2-pyridinylmethyl)-N′-(1H-imidazol-2-ylmethyl)-N′-(7-methoxy-1,2,3,4-tetrahydro-2-naphthalenyl)-1,4-benzenedimethanamine;
N-(2-pyridinylmethyl)-N′-(8-hydroxy-1,2,3,4-tetrahydro-2-naphthalenyl)-1,4-benzenedimethanamine;
N-(2-pyridinylmethyl)-N′-(1H-imidazol-2-ylmethyl)-N′-(8-hydroxy-1,2,3,4-tetrahydro-2-naphthalenyl)-1,4-benzenedimethanamine;
N-(2-pyridinylmethyl)-N′-(8-Fluoro-1,2,3,4-tetrahydro-2-naphthalenyl)-1,4-benzenedimethanamine;
N-(2-pyridinylmethyl)-N′-(1H-imidazol-2-ylmethyl)-N′-(8-Fluoro-1,2,3,4-tetrahydro-2-naphthalenyl)-1,4-benzenedimethanamine;
N-(2-pyridinylmethyl)-N′-(5,6,7,8-tetrahydro-7-quinolinyl)-1,4-benzenedimethanamine;
N-(2-pyridinylmethyl)-N′-(1H-imidazol-2-ylmethyl)-N′-(5,6,7,8-tetrahydro-7-quinolinyl)-1,4-benzenedimethanamine;
N-(2-pyridinylmethyl)-N′-[2-[(2-naphthalenylmethyl)amino]ethyl]-N′-(5,6,7,8-tetrahydro-8-quinolinyl)-1,4-benzenedimethanamine;
N-(2-pyridinylmethyl)-N′-[2-(isobutylamino)ethyl]-N′-(5,6,7,8-tetrahydro-8-quinolinyl)-1,4-benzenedimethanamine;
N-(2-pyridinylmethyl)-N′-[2-[(2-pyridinylmethyl)amino]ethyl]-N′-(5,6,7,8-tetrahydro-8-quinolinyl)-1,4-benzenedimethanamine;
N-(2-pyridinylmethyl)-N′-[2-[(2-furanylmethyl)amino]ethyl]-N′-(5,6,7,8-tetrahydro-8-quinolinyl)-1,4-benzenedimethanamine;
N-(2-pyridinylmethyl)-N′-(2-guanidinoethyl)-N′-(5,6,7,8-tetrahydro-8-quinolinyl)-1,4-benzenedimethanamine;
N-(2-pyridinylmethyl)-N′-[2-[bis-[(2-methoxy)phenylmethyl]amino]ethyl]-N′-(5,6,7,8-tetrahydro-8-quinolinyl)-1,4-benzene dimethanamine;
N-(2-pyridinylmethyl)-N′-[2-[(1H-imidazol-4-ylmethyl)amino]ethyl]-N′-(5,6,7,8-tetrahydro-8-quinolinyl)-1,4-benzene dimethanamine;
N-(2-pyridinylmethyl)-N′-[2-[(1H-imidazol-2-ylmethyl)amino]ethyl]-N′-(5,6,7,8-tetrahydro-8-quinolinyl)-1,4-benzenedimethanamine;
N-(2-pyridinylmethyl)-N′-[2-(phenylureido)ethyl]-N′-(5,6,7,8-tetrahydro-8-quinolinyl)-1,4-benzenedimethanamine;
N-(2-pyridinylmethyl)-N′-[[N″-(n-butyl)carboxamido]methyl]-N′-(5,6,7,8-tetrahydro-8-quinolinyl)-1,4-benzenedimethanamine;
N-(2-pyridinylmethyl)-N′-(carboxamidomethyl)-N′-(5,6,7,8-tetrahydro-8-quinolinyl)-1,4-benzenedimethanamine;
N-(2-pyridinylmethyl)-N′-[(N″-phenyl)carboxamidomethyl]-N′-(5,6,7,8-tetrahydro-8-quinolinyl)-1,4-benzenedimethanamine;
N-(2-pyridinylmethyl)-N′-(carboxymethyl)-N′-(5,6,7,8-tetrahydro-8-quinolinyl)-1,4-benzenedimethanamine;
N-(2-pyridinylmethyl)-N′-(phenylmethyl)-N′-(5,6,7,8-tetrahydro-8-quinolinyl)-1,4-benzenedimethanamine;
N-(2-pyridinylmethyl)-N′-(1H-benzimidazol-2-ylmethyl)-N′-(5,6,7,8-tetrahydro-8-quinolinyl)-1,4-benzenedimethanamine;
N-(2-pyridinylmethyl)-N′-(5,6-dimethyl-1H-benzimidazol-2-ylmethyl)-N′-(5,6,7,8-tetrahydro-8-quinolinyl)-1,4-benzenedimethanamine (hydrobromide salt);
N-(2-pyridinylmethyl)-N′-(5-nitro-1H-benzimidazol-2-ylmethyl)-N′-(5,6,7,8-tetrahydro-8-quinolinyl)-1,4-benzenedimethanamine;
N-(2-pyridinylmethyl)-N′-[(1H)-5-azabenzimidazol-2-ylmethyl]-N′-(5,6,7,8-tetrahydro-8-quinolinyl)-1,4-benzenedimethanamine;
N-(2-pyridinylmethyl)-N-(4-phenyl-1H-imidazol-2-ylmethyl)-N′-(5,6,7,8-tetrahydro-8-quinolinyl)-1,4-benzenedimethanamine;
N-(2-pyridinylmethyl)-N″-[2-(2-pyridinyl)ethyl]-N′-(5,6,7,8-tetrahydro-8-quinolinyl)-1,4-benzenedimethanamine;
N-(2-pyridinylmethyl)-N′-(2-benzoxazolyl)-N′-(5,6,7,8-tetrahydro-8-quinolinyl)-1,4-benzenedimethanamine;
N-(2-pyridinylmethyl)-N′-(trans-2-aminocyclohexyl)-N′-(5,6,7,8-tetrahydro-8-quinolinyl)-1,4-benzenedimethanamine;
N-(2-pyridinylmethyl)-N′-(2-phenylethyl)-N′-(5,6,7,8-tetrahydro-8-quinolinyl)-1,4-benzenedimethanamine;
N-(2-pyridinylmethyl)-N′-(3-phenylpropyl)-N′-(5,6,7,8-tetrahydro-8-quinolinyl)-1,4-benzenedimethanamine;
N-(2-pyridinylmethyl)-N′-(trans-2-aminocyclopentyl)-N′-(5,6,7,8-tetrahydro-8-quinolinyl)-1,4-benzenedimethanamine;
N-[[4-[[(2-pyridinylmethyl)amino]methyl]phenyl]methyl]-N-(5,6,7,8-tetrahydro-8-quinolinyl)-glycinamide;
N-[[4-[[(2-pyridinylmethyl)amino]methyl]phenyl]methyl]-N-(5,6,7,8-tetrahydro-8-quinolinyl)-(L)-alaninamide;
N-[[4-[[(2-pyridinylmethyl)amino]methyl]phenyl]methyl]-N-(5,6,7,8-tetrahydro-8-quinolinyl)-(L)-aspartamide;
N-[[4-[[(2-pyridinylmethyl)amino]methyl]phenyl]methyl]-N-(5,6,7,8-tetrahydro-8-quinolinyl)-pyrazinamide;
N-[[4-[[(2-pyridinylmethyl)amino]methyl]phenyl]methyl]-N-(5,6,7,8-tetrahydro-8-quinolinyl)-(L)-prolinamide;
N-[[4-[[(2-pyridinylmethyl)amino]methyl]phenyl]methyl]-N-(5,6,7,8-tetrahydro-8-quinolinyl)-(L)-lysinamide;
N-[[4-[[(2-pyridinylmethyl)amino]methyl]phenyl]methyl]-N-(5,6,7,8-tetrahydro-8-quinolinyl)-benzamide;
N-[[4-[[(2-pyridinylmethyl)amino]methyl]phenyl]methyl]-N-(5,6,7,8-tetrahydro-8-quinolinyl)-picolinamide;
N′-Benzyl-N-[[4-[[(2-pyridinylmethyl)amino]methyl]phenyl]methyl]-N-(5,6,7,8-tetrahydro-8-quinolinyl)-urea;
N′-phenyl-N-[[4-[[(2-pyridinylmethyl)amino]methyl]phenyl]methyl]-N-(5,6,7,8-tetrahydro-8-quinolinyl)-urea;
N-(6,7,8,9-tetrahydro-5H-cyclohepta[bacteriapyridin-9-yl)-4-[[(2-pyridinylmethyl)amino]methyl]benzamide;
N-(5,6,7,8-tetrahydro-8-quinolinyl)-4-[[(2-pyridinylmethyl)amino]methyl]benzamide;
N,N′-bis(2-pyridinylmethyl)-N′-(5,6,7,8-tetrahydro-8-quinolinyl)-1,4-benzenedimethanamine;
N,N′-bis(2-pyridinylmethyl)-N′-(6,7,8,9-tetrahydro-5H-cyclohepta[bacteriapyridin-9-yl)-1,4-benzenedimethanamine;
N,N′-bis(2-pyridinylmethyl)-N′-(6,7-dihydro-5H-cyclopenta[bacteriapyridin-7-yl)-1,4-benzenedimethanamine;
N,N′-bis(2-pyridinylmethyl)-N′-(1,2,3,4-tetrahydro-1-naphthalenyl)-1,4-benzenedimethanamine;
N,N′-bis(2-pyridinylmethyl)-N′-[(5,6,7,8-tetrahydro-8-quinolinyl)methyl]-1,4-benzenedimethanamine;
N,N′-bis(2-pyridinylmethyl)-N′[(6,7-dihydro-5H-cyclopenta[bacteriapyridin-7-yl)methyl]-1,4-benzenedimethanamine;
N-(2-pyridinylmethyl)-N-(2-methoxyethyl)-N′-(5,6,7,8-tetrahydro-8-quinolinyl)-1,4-benzenedimethanamine;
N-(2-pyridinylmethyl)-N-[2-(4-methoxyphenyl)ethyl]-N′-(5,6,7,8-tetrahydro-8-quinolinyl)-1,4-benzenedimethanamine;
N,N′-bis(2-pyridinylmethyl)-1,4-(5,6,7,8-tetrahydro-8-quinolinyl)benzenedimethanamine;
N-[(2,3-dimethoxyphenyl)methyl]-N′-(2-pyridinylmethyl)-N-(5,6,7,8-tetrahydro-8-quinolinyl)-1,4-benzenedimethanamine;
N,N′-bis(2-pyridinylmethyl)-N-[1-(N″-phenyl-N″-methylureido)-4-piperidinyl]-1,3-benzenedimethanamine;
N,N′-bis(2-pyridinylmethyl)-N-[N″-p-toluenesulfonylphenylalanyl)-4-piperidinyl]-1,3-benzenedimethanamine;
N,N′-bis(2-pyridinylmethyl)-N-[1-[3-(2-chlorophenyl)-5-methyl-isoxazol-4-oyl]-4-piperidinyl]-1,3-benzenedimethanamine;
N-[(2-hydroxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(4-cyanophenyl)methyl]-N′-(2-pyridinylmethyl)-N-(6,7,8,9-tetrahydro-5H-cyclohepta[bacteriapyridin-9-yl)-1,4-benzenedimethanamine;
N-[(4-cyanophenyl)methyl]-N′-(2-pyridinylmethyl)-N-(5,6,7,8-tetrahydro-8-quinolinyl)-1,4-benzenedimethanamine;
N-[(4-acetamidophenyl)methyl]-N′-(2-pyridinylmethyl)-N-(5,6,7,8-tetrahydro-8-quinolinyl)-1,4-benzenedimethanamine;
N-[(4-phenoxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(1-methyl-2-carboxamido)ethyl]-N,N′-bis(2-pyridinylmethyl)-1,3-benzenedimethanamine;
N-[(4-benzyloxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(thiophene-2-yl)methyl]-N′-(2-pyridinylmethyl)-N-(6,7,8,9-tetrahydro-5H-cyclohepta[bacteriapyridin-9-yl)-1,4-benzenedimethanamine;
N-[1-(benzyl)-3-pyrrolidinyl]-N,N′-bis(2-pyridinylmethyl)-1,3-benzenedimethanamine;
N-[[1-methyl-3-(pyrazol-3-yl)]propyl]-N,N′-bis(2-pyridinylmethyl)-1,3-benzenedimethanamine;
N-[1-(phenyl)ethyl]-N,N′-bis(2-pyridinylmethyl)-1,3-benzenedimethanamine;
N-[(3,4-methylenedioxyphenyl)methyl]-N′-(2-pyridinylmethyl)-N-(6,7,8,9-tetrahydro-5H-cyclohepta[b]pyridin-9-yl)-1,4-benzenedimethanamine;
N-[1-benzyl-3-carboxymethyl-4-piperidinyl]-N,N′-bis(2-pyridinylmethyl)-1,3-benzenedimethanamine;
N-[(3,4-methylenedioxyphenyl)methyl]-N′-(2-pyridinylmethyl)-N-(5,6,7,8-tetrahydro-8-quinolinyl)-1,4-benzenedimethanamine;
N-(3-pyridinylmethyl)-N′-(2-pyridinylmethyl)-N-(6,7,8,9-tetrahydro-5H-cyclohepta pyridin-9-yl)-1,4-benzenedimethanamine;
N-[[1-methyl-2-(2-tolyl)carboxamido]ethyl]-N,N′-bis(2-pyridinylmethyl)-1,3-benzenedimethanamine;
N-[(1,5-dimethyl-2-phenyl-3-pyrazolinone-4-yl)methyl]-N′-(2-pyridinylmethyl)-N-(5,6,7,8-tetrahydro-8-quinolinyl)-1,4-benzenedimethanamine;
N-[(4-propoxyphenyl)methyl]-N′-(2-pyridinylmethyl)-N-(6,7,8,9-tetrahydro-5H-cyclohepta[b]pyridin-9-yl)-1,4-benzenedimethanamine;
N-(1-phenyl-3,5-dimethylpyrazolin-4-ylmethyl)-N′-(2-pyridinylmethyl)-N-(5,6,7,8-tetrahydro-8-quinolinyl)-1,4-benzenedimethanamine;
N-[1H-imidazol-4-ylmethyl]-N,N′-bis(2-pyridinylmethyl)-1,3-benzenedimethanamine;
N-[(3-methoxy-4,5-methylenedioxyphenyl)methyl]-N′-(2-pyridinylmethyl)-N-(6,7,8,9-tetrahydro-5H-cyclohepta[b]pyridin-9-yl)-1,4-benzenedimethanamine;
N-[(3-cyanophenyl)methyl]-N′-(2-pyridinylmethyl)-N-(6,7,8,9-tetrahydro-5H-cyclohepta[b]pyridin-9-yl)-1,4-benzenedimethanamine;
N-[(3-cyanophenyl)methyl]-N′-(2-pyridinylmethyl)-N-(5,6,7,8-tetrahydro-8-quinolinyl)-1,4-benzenedimethanamine;
N-(5-ethylthiophene-2-ylmethyl)-N′-(2-pyridinylmethyl)-N-(6,7,8,9-tetrahydro-5H-cyclohepta[b]pyridin-9-yl)-1,4-benzenedimethanamine;
N-(5-ethylthiophene-2-ylmethyl)-N′-(2-pyridinylmethyl)-N-(5,6,7,8-tetrahydro-8-quinolinyl)-1,4-benzenedimethanamine;
N-[(2,6-difluorophenyl)methyl]-N′-(2-pyridinylmethyl)-N-(6,7,8,9-tetrahydro-5H-cyclohepta[b]pyridin-9-yl)-1,4-benzenedimethanamine;
N-[(2,6-difluorophenyl)methyl]-N′-(2-pyridinylmethyl)-N-(5,6,7,8-tetrahydro-8-quinolinyl)-1,4-benzenedimethanamine;
N-[(2-difluoromethoxyphenyl)methyl]-N′-(2-pyridinylmethyl)-N-(6,7,8,9-tetrahydro-5H-cyclohepta[b]pyridin-9-yl)-1,4-benzenedimethanamine;
N-(2-difluoromethoxyphenylmethyl)-N′-(2-pyridinylmethyl)-N-(5,6,7,8-tetrahydro-8-quinolinyl)-1,4-benzenedimethanamine;
N-(1,4-benzodioxan-6-ylmethyl)-N′-(2-pyridinylmethyl)-N-(6,7,8,9-tetrahydro-5H-cyclohepta[b]pyridin-9-yl)-1,4-benzenedimethanamine;
N,N′-bis(2-pyridinylmethyl)-N-[1-(N″-phenyl-N″-methylureido)-4-piperidinyl]-1,4-benzenedimethanamine;
N,N′-bis(2-pyridinylmethyl)-N-[N″-p-toluenesulfonylphenylalanyl)-4-piperidinyl]-1,4-benzenedimethanamine;
N-[1-(3-pyridinecarboxamido)-4-piperidinyl]-N,N′-bis(2-pyridinylmethyl)-1,4-benzenedimethanamine;
N-[1-(cyclopropylcarboxamido)-4-piperidinyl]-N,N′-bis(2-pyridinylmethyl)-1,4-benzenedimethanamine;
N-[1-(1-phenylcyclopropylcarboxamido)-4-piperidinyl]-N,N′-bis(2-pyridinylmethyl)-1,4-benzenedimethanamine;
N-(1,4-benzodioxan-6-ylmethyl)-N′-(2-pyridinylmethyl)-N-(5,6,7,8-tetrahydro-8-quinolinyl)-1,4-benzenedimethanamine;
N-[1-[3-(2-chlorophenyl)-5-methyl-isoxazol-4-carboxamido]-4-piperidinyl]-N,N′-bis(2-pyridinylmethyl)-1,4-benzenedimethanamine;
N-[1-(2-thiomethylpyridine-3-carboxamido)-4-piperidinyl]-N,N′-bis(2-pyridinylmethyl)-1,4-benzenedimethanamine;
N-[(2,4-difluorophenyl)methyl]-N′-(2-pyridinylmethyl)-N-(5,6,7,8-tetrahydro-8-quinolinyl)-1,4-benzenedimethanamine;
N-(1-methylpyrrol-2-ylmethyl)-N′-(2-pyridinylmethyl)-N-(5,6,7,8-tetrahydro-8-quinolinyl)-1,4-benzenedimethanamine;
N-[(2-hydroxyphenyl)methyl]-N′-(2-pyridinylmethyl)-N-(5,6,7,8-tetrahydro-8-quinolinyl)-1,4-benzenedimethanamine;
N-[(3-methoxy-4,5-methylenedioxyphenyl)methyl]-N′-(2-pyridinylmethyl)-N-(5,6,7,8-tetrahydro-8-quinolinyl)-1,4-benzenedimethanamine;
N-(3-pyridinylmethyl)-N′-(2-pyridinylmethyl)-N-(5,6,7,8-tetrahydro-8-quinolinyl)-1,4-benzenedimethanamine;
N-[2-(N″-morpholinomethyl)-1-cyclopentyl]-N,N′-bis(2-pyridinylmethyl)-1,4-benzenedimethanamine;
N-[(1-methyl-3-piperidinyl)propyl]-N,N′-bis(2-pyridinylmethyl)-1,4-benzenedimethanamine;
N-(1-methylbenzimidazol-2-ylmethyl)-N′-(2-pyridinylmethyl)-N-(5,6,7,8-tetrahydro-8-quinolinyl)-1,4-benzenedimethanamine;
N-[1-(benzyl)-3-pyrrolidinyl]-N,N′-bis(2-pyridinylmethyl)-1,4-benzenedimethanamine;
N-[[(1-phenyl-3-(N″-morpholino)]propyl]-N,N′-bis(2-pyridinylmethyl)-1,4-benzenedimethanamine;
N-[1-(iso-propyl)-4-piperidinyl]-N,N′-bis(2-pyridinylmethyl)-1,4-benzenedimethanamine;
N-[1-(ethoxycarbonyl)-4-piperidinyl]-N′-(2-pyridinylmethyl)-N-(5,6,7,8-tetrahydro-8-quinolinyl)-1,4-benzenedimethanamine;
N-[(1-methyl-3-pyrazolyl)propyl]-N′-(2-pyridinylmethyl)-N-(5,6,7,8-tetrahydro-8-quinolinyl)-1,4-benzenedimethanamine;
N-[1-methyl-2-(N″,N″-diethylcarboxamido)ethyl]-N,N′-bis(2-pyridinylmethyl)-1,4-benzenedimethanamine;
N-[(1-methyl-2-phenylsulfonyl)ethyl]-N′-(2-pyridinylmethyl)-N-(5,6,7,8-tetrahydro-8-quinolinyl)-1,4-benzenedimethanamine;
N-[(2-chloro-4,5-methylenedioxyphenyl)methyl]-N′-(2-pyridinylmethyl)-N-(5,6,7,8-tetrahydro-8-quinolinyl)-1,4-benzenedimethanamine;
N-[1-methyl-2-[N″-(4-chlorophenyl)carboxamido]ethyl]-N′-(2-pyridinylmethyl)-N-(5,6,7,8-tetrahydro-8-quinolinyl)-1,4-benzenedimethanamine;
N-(1-acetoxyindol-3-ylmethyl)-N′-(2-pyridinylmethyl)-N-(6,7,8,9-tetrahydro-5H-cyclohepta[b]pyridin-9-yl)-1,4-benzenedimethanamine;
N-[(3-benzyloxy-4-methoxyphenyl)methyl]-N′-(2-pyridinylmethyl)-N-(6,7,8,9-tetrahydro-5H-cyclohepta[b]pyridin-9-yl)-1,4-benzenedimethanamine;
N-(3-quinolylmethyl)-N′-(2-pyridinylmethyl)-N-(5,6,7,8-tetrahydro-8-quinolinyl)-1,4-benzenedimethanamine;
N-[(8-hydroxy)-2-quinolylmethyl]-N′-(2-pyridinylmethyl)-N-(6,7,8,9-tetrahydro-5H-cyclohepta[b]pyridin-9-yl)-1,4-benzenedimethanamine;
N-(2-quinolylmethyl)-N′-(2-pyridinylmethyl)-N-(6,7,8,9-tetrahydro-5H-cyclohepta[b]pyridin-9-yl)-1,4-benzenedimethanamine;
N-[(4-acetamidophenyl)methyl]-N′-(2-pyridinylmethyl)-N-(6,7,8,9-tetrahydro-5H-cyclohepta[b]pyridin-9-yl)-1,4-benzenedimethanamine;
N-[1H-imidazol-2-ylmethyl]-N,N′-bis(2-pyridinylmethyl)-1,4-benzenedimethanamine;
N-(3-quinolylmethyl)-N′-(2-pyridinylmethyl)-N-(6,7,8,9-tetrahydro-5H-cyclohepta[b]pyridin-9-yl)-1,4-benzenedimethanamine;
N-(2-thiazolylmethyl)-N′-(2-pyridinylmethyl)-N-(6,7,8,9-tetrahydro-5H-cyclohepta[b]pyridin-9-yl)-1,4-benzenedimethanamine;
N-(4-pyridinylmethyl)-N′-(2-pyridinylmethyl)-N-(6,7,8,9-tetrahydro-5H-cyclohepta[b]pyridin-9-yl)-1,4-benzenedimethanamine;
N-[(5-benzyloxy)benzo[b]pyridin-3-ylmethyl]-N,N′-bis(2-pyridinylmethyl)-1,4-benzenedimethanamine;
N-(1-methylpyrazol-2-ylmethyl)-N′-(2-pyridinylmethyl)-N-(6,7,8,9-tetrahydro-5H-cyclohepta[b]pyridin-9-yl)-1,4-benzenedimethanamine;
N-[(4-methyl)-1H-imidazol-5-ylmethyl]-N,N′-bis(2-pyridinylmethyl)-1,4-benzenedimethanamine;
N-[[(4-dimethylamino)-1-napthalenyl]methyl]-N,N′-bis(2-pyridinylmethyl)-1,4-benzenedimethanamine;
N-[1,5-dimethyl-2-phenyl-3-pyrazolinone-4-ylmethyl]-N,N′-bis(2-pyridinylmethyl)-1,4-benzenedimethanamine;
N-[1-[(1-acetyl-2-(R)-prolinyl]-4-piperidinyl]-N-[2-(2-pyridinyl)ethyl]-N′-(2-pyridinylmethyl)-1,3-benzenedimethanamine;
N-[1-[2-acetamidobenzoyl-4-piperidinyl]-4-piperidinyl]-N-[2-(2-pyridinyl)ethyl]-N′-(2-pyridinylmethyl)-1,3-benzenedimethanamine;
N-[(2-cyano-2-phenyl)ethyl]-N′-(2-pyridinylmethyl)-N-(6,7,8,9-tetrahydro-5H-cyclohepta[b]pyridin-9-yl)-1,4-benzenedimethanamine;
N-[(N″-acetyltryptophanyl)-4-piperidinyl]-N-[2-(2-pyridinyl)ethyl]-N′-(2-pyridinylmethyl)-1,3-benzenedimethanamine;
N-[(N″-benzoylvalinyl)-4-piperidinyl]-N-[2-(2-pyridinyl)ethyl]-N′-(2-pyridinylmethyl)-1,3-benzenedimethanamine;
N-[(4-dimethylaminophenyl)methyl]-N′-(2-pyridinylmethyl)-N-(6,7,8,9-tetrahydro-5H-cyclohepta[b]pyridin-9-yl)-1,4-benzenedimethanamine;
N-(4-pyridinylmethyl)-N′-(2-pyridinylmethyl)-N-(5,6,7,8-tetrahydro-8-quinolinyl)-1,4-benzenedimethanamine;
N-(1-methylbenzimadazol-2-ylmethyl)-N′-(2-pyridinylmethyl)-N-(6,7,8,9-tetrahydro-5H-cyclohepta[b]pyridin-9-yl)-1,4-benzenedimethanamine;
N-[1-butyl-4-piperidinyl]-N-[2-(2-pyridinyl)ethyl]-N′-(2-pyridinylmethyl)-1,3-benzenedimethanamine;
N-[1-benzoyl-4-piperidinyl]-N-[2-(2-pyridinyl)ethyl]-N′-(2-pyridinylmethyl)-1,3-benzenedimethanamine;
N-[1-(benzyl)-3-pyrrolidinyl]-N-[2-(2-pyridinyl)ethyl]-N′-(2-pyridinylmethyl)-1,3-benzenedimethanamine;
N-[(1-methyl)benzo[b]pyrrol-3-ylmethyl]-N-[2-(2-pyridinyl)ethyl]-N′-(2-pyridinylmethyl)-1,3-benzenedimethanamine;
N-[1H-imidazol-4-ylmethyl]-N-[2-(2-pyridinyl)ethyl]-N′-(2-pyridinylmethyl)-1,3-benzenedimethanamine;
N-[1-(benzyl)-4-piperidinyl]-N-[2-(2-pyridinyl)ethyl]-N′-(2-pyridinylmethyl)-1,4-benzenedimethanamine;
N-[1-methylbenzimidazol-2-ylmethyl]-N-[2-(2-pyridinyl)ethyl]-N′-(2-pyridinylmethyl)-1,4-benzenedimethanamine;
N-[(2-phenyl)benzo[b]pyrrol-3-ylmethyl]-N-[2-(2-pyridinyl)ethyl]-N′-(2-pyridinylmethyl)-1,4-benzenedimethanamine;
N-[(6-methylpyridin-2-yl)methyl]-N′-(2-pyridinylmethyl)-N-(5,6,7,8-tetrahydro-8-quinolinyl)-1,4-benzenedimethanamine;
N-(3-methyl-1H-pyrazol-5-ylmethyl)-N′-(2-pyridinylmethyl)-N-(5,6,7,8-tetrahydro-8-quinolinyl)-1,3-benzenedimethanamine;
N-[(2-methoxyphenyl)methyl]-N′-(2-pyridinylmethyl)-N-(5,6,7,8-tetrahydro-8-quinolinyl)-1,3-benzenedimethanamine;
N-[(2-ethoxyphenyl)methyl]-N′-(2-pyridinylmethyl)-N-(6,7,8,9-tetrahydro-5H-cyclohepta[b]pyridin-9-yl)-1,3-benzenedimethanamine;
N-(benzyloxyethyl)-N′-(2-pyridinylmethyl)-N-(5,6,7,8-tetrahydro-8-quinolinyl)-1,3-benzenedimethanamine;
N-[(2-ethoxy-1-naphthalenyl)methyl]-N′-(2-pyridinylmethyl)-N-(5,6,7,8-tetrahydro-8-quinolinyl)-1,3-benzenedimethanamine;
N-[(6-methylpyridin-2-yl)methyl]-N′-(2-pyridinylmethyl)-N-(5,6,7,8-tetrahydro-8-quinolinyl)-1,3-benzenedimethanamine;
1-[[4-[[(2-pyridinylmethyl)amino]methyl]phenyl]methyl]guanidine;
N-(2-pyridinylmethyl)-N-(8-methyl-8-azabicyclo[3.2.1]octan-3-yl)-1,4-benzenedimethanamine;
1-[[4-[[(2-pyridinylmethyl)amino]methyl]phenyl]methyl]homopiperazine;
1-[[3-[[(2-pyridinylmethyl)amino]methyl]phenyl]methyl]homopiperazine;
trans and cis-1-[[4-[[(2-pyridinylmethyl)amino]methyl]phenyl]methyl]-3,5-piperidinediamine;
N,N′-[1,4-Phenylenebis(methylene)]bis-4-(2-pyrimidyl)piperazine;
1-[[4-[[(2-pyridinylmethyl)amino]methyl]phenyl]methyl]-1-(2-pyridinyl)methylamine;
2-(2-pyridinyl)-5-[[(2-pyridinylmethyl)amino]methyl]-1,2,3,4-tetrahydroisoquino line;
1-[[4-[[(2-pyridinylmethyl)amino]methyl]phenyl]methyl]-3,4-diaminopyrrolidine;
1-[[4-[[(2-pyridinylmethyl)amino]methyl]phenyl]methyl]-3,4-diacetylaminopyrrolidine;
8-[[4-[[(2-pyridinylmethyl)amino]methyl]phenyl]methyl]-2,5,8-triaza-3-oxabicyclo[4.3.0]nonan;
8-[[4-[[(2-pyridinylmethyl)amino]methyl]phenyl]methyl]-2,5,8-triazabicyclo[4.3.0]nonane;
(4-Aminomethyl-pyridin-3-ylmethyl)-(1H-benzoimidazol-2-ylmethyl)-(5,6,7,8-tetrahydro-quinolin-8-yl)-amine;
(3-Aminomethyl-pyridin-4-ylmethyl)-(1H-benzoimidazol-2-ylmethyl)-(5,6,7,8-tetrahydro-quinolin-8-yl)-amine;
1-(3-Aminomethyl-4-{[(1H-benzoimidazol-2-ylmethyl)-(5,6,7,8-tetrahydro-quinolin-8-yl)-amino]-methyl}-phenyl)-ethanone;
1-(5-Aminomethyl-2-{[(1H-benzoimidazol-2-ylmethyl)-(5,6,7,8-tetrahydro-quinolin-8-yl)-amino]-methyl}-phenyl)-ethanone;
3-Aminomethyl-4-{[(1H-benzoimidazol-2-ylmethyl)-(5,6,7,8-tetrahydro-quinolin-8-yl)-amino]-methyl}-benzenesulfonamide;
5-Aminomethyl-2-{[(1H-benzoimidazol-2-ylmethyl)-(5,6,7,8-tetrahydro-quinolin-8-yl)-amino]-methyl}-benzenesulfonamide;
N-(3-Aminomethyl-4-{[(1H-benzoimidazol-2-ylmethyl)-(5,6,7,8-tetrahydro-quinolin-8-yl)-amino]-methyl}-benzyl)-hydroxylamine;
N-(5-Aminomethyl-2-{[(1H-benzoimidazol-2-ylmethyl)-(5,6,7,8-tetrahydro-quinolin-8-yl)-amino]-methyl}-benzyl)-hydroxylamine;
N-(3-Aminomethyl-4-{[(1H-benzoimidazol-2-ylmethyl)-(5,6,7,8-tetrahydro-quinolin-8-yl)-amino]-methyl}-benzyl)-O-methyl-hydroxylamine;
N-(5-Aminomethyl-2-{[(1H-benzoimidazol-2-ylmethyl)-(5,6,7,8-tetrahydro-quinolin-8-yl)-amino]-methyl}-benzyl)-O-methyl-hydroxylamine;
(4-Aminomethyl-2-methoxymethyl-benzyl)-(1H-benzoimidazol-2-ylmethyl)-(5,6,7,8-tetrahydro-quinolin-8-yl)-amine;
(2-Aminomethyl-4-methoxymethyl-benzyl)-(1H-benzoimidazol-2-ylmethyl)-(5,6,7,8-tetrahydro-quinolin-8-yl)-amine;
N-(2-{[(1H-Benzoimidazol-2-ylmethyl)-(5,6,7,8-tetrahydro-quinolin-8-yl)-amino]-methyl}-benzyl)-formamide;
N-(4-{[(1H-Benzoimidazol-2-ylmethyl)-(5,6,7,8-tetrahydro-quinolin-8-yl)-amino]-methyl}-benzyl)-formamide;
N-(2-{[(1H-Benzoimidazol-2-ylmethyl)-(5,6,7,8-tetrahydro-quinolin-8-yl)-amino]-methyl}-benzyl)-hydroxylamine;
(1H-Benzoimidazol-2-ylmethyl)-(2,6-bis-aminomethyl-benzyl)-(5,6,7,8-tetrahydro-quinolin-8-yl)-amine;
(3-Aminomethyl-2-{[(1H-benzoimidazol-2-ylmethyl)-(5,6,7,8-tetrahydro-quinolin-8-yl)-amino]-methyl}-phenyl)-methanol;
(2-Aminomethyl-6-methoxymethyl-benzyl)-(1H-benzoimidazol-2-ylmethyl)-(5,6,7,8-tetrahydro-quinolin-8-yl)-amine;
N-(3-Aminomethyl-2-{[(1H-benzoimidazol-2-ylmethyl)-(5,6,7,8-tetrahydro-quinolin-8-yl)-amino]-methyl}-benzyl)-hydroxylamine;
N-(3-Aminomethyl-2-{[(1H-benzoimidazol-2-ylmethyl)-(5,6,7,8-tetrahydro-quinolin-8-yl)-amino]-methyl}-benzyl)-O-methyl-hydroxylamine;
[2-Aminomethyl-4-(1H-imidazol-2-yl)-benzyl]-(1H-benzoimidazol-2-ylmethyl)-(5,6,7,8-tetrahydro-quinolin-8-yl)-amine;
[2-Aminomethyl-4-(1-methyl-1H-imidazol-2-yl)-benzyl]-(1H-benzoimidazol-2-ylmethyl)-(5,6,7,8-tetrahydro-quinolin-8-yl)-amine;
[2-Aminomethyl-4-(2H-pyrazol-3-yl)-benzyl]-(1H-benzoimidazol-2-ylmethyl)-(5,6,7,8-tetrahydro-quinolin-8-yl)-amine;
[2-Aminomethyl-4-(1-methyl-1H-pyrazol-3-yl)-benzyl]-(1H-benzoimidazol-2-ylmethyl)-(5,6,7,8-tetrahydro-quinolin-8-yl)-amine;
[2-Aminomethyl-4-(1H-[1,2,4]triazol-3-yl)-benzyl]-(1H-benzoimidazol-2-ylmethyl)-(5,6,7,8-tetrahydro-quinolin-8-yl)-amine;
[2-Aminomethyl-4-(1-methyl-1H-[1,2,4]triazol-3-yl)-benzyl]-(1H-benzoimidazol-2-ylmethyl)-(5,6,7,8-tetrahydro-quinolin-8-yl)-amine;
(2-Aminomethyl-4-oxazol-2-yl-benzyl)-(1H-benzoimidazol-2-ylmethyl)-(5,6,7,8-tetrahydro-quinolin-8-yl)-amine;
(2-Aminomethyl-4-furan-2-yl-benzyl)-(1H-benzoimidazol-2-ylmethyl)-(5,6,7,8-tetrahydro-quinolin-8-yl)-amine;
[2-Aminomethyl-4-(tetrahydro-furan-2-yl)-benzyl]-(1H-benzoimidazol-2-ylmethyl)-(5,6,7,8-tetrahydro-quinolin-8-yl)-amine;
(2-Aminomethyl-4-thiazol-2-yl-benzyl)-(1H-benzoimidazol-2-ylmethyl)-(5,6,7,8-tetrahydro-quinolin-8-yl)-amine;
[2-Aminomethyl-4-(1H-tetrazol-5-yl)-benzyl]-(1H-benzoimidazol-2-ylmethyl)-(5,6,7,8-tetrahydro-quinolin-8-yl)-amine;
[2-Aminomethyl-4-(2-methyl-2H-tetrazol-5-yl)-benzyl]-(1H-benzoimidazol-2-ylmethyl)-(5,6,7,8-tetrahydro-quinolin-8-yl)-amine;
(2-Aminomethyl-4-pyridin-2-yl-benzyl)-(1H-benzoimidazol-2-ylmethyl)-(5,6,7,8-tetrahydro-quinolin-8-yl)-amine;
(2-Aminomethyl-4-piperidin-2-yl-benzyl)-(1H-benzoimidazol-2-ylmethyl)-(5,6,7,8-tetrahydro-quinolin-8-yl)-amine;
(4-Aminomethyl-3-{[(1H-benzoimidazol-2-ylmethyl)-(5,6,7,8-tetrahydro-quinolin-8-yl)-amino]-methyl}-phenyl)-methanol;
(2-Aminomethyl-5-methoxymethyl-benzyl)-(1H-benzoimidazol-2-ylmethyl)-(5,6,7,8-tetrahydro-quinolin-8-yl)-amine;
(4-Aminomethyl-5-{[(1H-benzoimidazol-2-ylmethyl)-(5,6,7,8-tetrahydro-quinolin-8-yl)-amino]-methyl}-pyridin-2-yl)-methanol;
(4-Aminomethyl-6-methoxymethyl-pyridin-3-ylmethyl)-(1H-benzoimidazol-2-ylmethyl)-(5,6,7,8-tetrahydro-quinolin-8-yl)-amine;
(1H-Benzoimidazol-2-ylmethyl)-(4,6-bis-aminomethyl-pyridin-3-ylmethyl)-(5,6,7,8-tetrahydro-quinolin-8-yl)-amine;
(4-Allylaminomethyl-2-aminomethyl-benzyl)-(1H-benzoimidazol-2-ylmethyl)-(5,6,7,8-tetrahydro-quinolin-8-yl)-amine;
(2-Allylaminomethyl-4-aminomethyl-benzyl)-(1H-benzoimidazol-2-ylmethyl)-(5,6,7,8-tetrahydro-quinolin-8-yl)-amine;
(2-Aminomethyl-4-cyclopropylaminomethyl-benzyl)-(1H-benzoimidazol-2-ylmethyl)-(5,6,7,8-tetrahydro-quinolin-8-yl)-amine;
(4-Aminomethyl-2-cyclopropylaminomethyl-benzyl)-(1H-benzoimidazol-2-ylmethyl)-(5,6,7,8-tetrahydro-quinolin-8-yl)-amine;
(2-Aminomethyl-5-chloro-benzyl)-(1H-benzoimidazol-2-ylmethyl)-(5,6,7,8-tetrahydro-quinolin-8-yl)-amine;
(2-Aminomethyl-5-bromo-benzyl)-(1H-benzoimidazol-2-ylmethyl)-(5,6,7,8-tetrahydro-quinolin-8-yl)-amine;
(2-Aminomethyl-5-nitro-benzyl)-(1H-benzoimidazol-2-ylmethyl)-(5,6,7,8-tetrahydro-quinolin-8-yl)-amine;
4-Aminomethyl-3-{[(1H-benzoimidazol-2-ylmethyl)-(5,6,7,8-tetrahydro-quinolin-8-yl)-amino]-methyl}-benzonitrile;
(5-Amino-2-aminomethyl-benzyl)-(1H-benzoimidazol-2-ylmethyl)-(5,6,7,8-tetrahydro-quinolin-8-yl)-amine;
(2-Aminomethyl-5-trifluoromethyl-benzyl)-(1H-benzoimidazol-2-ylmethyl)-(5,6,7,8-tetrahydro-quinolin-8-yl)-amine;
(2-Aminomethyl-4-fluoro-benzyl)-(1H-benzoimidazol-2-ylmethyl)-(5,6,7,8-tetrahydro-quinolin-8-yl)-amine;
(2-Aminomethyl-4-chloro-benzyl)-(1H-benzoimidazol-2-ylmethyl)-(5,6,7,8-tetrahydro-quinolin-8-yl)-amine;
(2-Aminomethyl-4-bromo-benzyl)-(1H-benzoimidazol-2-ylmethyl)-(5,6,7,8-tetrahydro-quinolin-8-yl)-amine;
(2-Aminomethyl-4-nitro-benzyl)-(1H-benzoimidazol-2-ylmethyl)-(5,6,7,8-tetrahydro-quinolin-8-yl)-amine;
3-Aminomethyl-4-{[(1H-benzoimidazol-2-ylmethyl)-(5,6,7,8-tetrahydro-quinolin-8-yl)-amino]-methyl}-benzonitrile;
(4-Amino-2-aminomethyl-benzyl)-(1H-benzoimidazol-2-ylmethyl)-(5,6,7,8-tetrahydro-quinolin-8-yl)-amine;
(2-Aminomethyl-4-trifluoromethyl-benzyl)-(1H-benzoimidazol-2-ylmethyl)-(5,6,7,8-tetrahydro-quinolin-8-yl)-amine;
(4-Aminomethyl-2-fluoro-benzyl)-(1H-benzoimidazol-2-ylmethyl)-(5,6,7,8-tetrahydro-quinolin-8-yl)-amine;
(4-Aminomethyl-2-chloro-benzyl)-(1H-benzoimidazol-2-ylmethyl)-(5,6,7,8-tetrahydro-quinolin-8-yl)-amine;
(4-Aminomethyl-2-bromo-benzyl)-(1H-benzoimidazol-2-ylmethyl)-(5,6,7,8-tetrahydro-quinolin-8-yl)-amine;
(4-Aminomethyl-2-nitro-benzyl)-(1H-benzoimidazol-2-ylmethyl)-(5,6,7,8-tetrahydro-quinolin-8-yl)-amine;
5-Aminomethyl-2-{[(1H-benzoimidazol-2-ylmethyl)-(5,6,7,8-tetrahydro-quinolin-8-yl)-amino]-methyl}-benzonitrile;
(2-Amino-4-aminomethyl-benzyl)-(1H-benzoimidazol-2-ylmethyl)-(5,6,7,8-tetrahydro-quinolin-8-yl)-amine;
(4-Aminomethyl-2-trifluoromethyl-benzyl)-(1H-benzoimidazol-2-ylmethyl)-(5,6,7,8-tetrahydro-quinolin-8-yl)-amine;
(5-Aminomethyl-thiophen-2-ylmethyl)-(1H-benzoimidazol-2-ylmethyl)-(5,6,7,8-tetrahydro-quinolin-8-yl)-amine;
(4-Aminomethyl-thiophen-3-ylmethyl)-(1H-benzoimidazol-2-ylmethyl)-(5,6,7,8-tetrahydro-quinolin-8-yl)-amine;
(4-Aminomethyl-furan-3-ylmethyl)-(1H-benzoimidazol-2-ylmethyl)-(5,6,7,8-tetrahydro-quinolin-8-yl)-amine;
(4-Aminomethyl-1H-pyrrol-3-ylmethyl)-(1H-benzoimidazol-2-ylmethyl)-(5,6,7,8-tetrahydro-quinolin-8-yl)-amine;
(4-Aminomethyl-1-methyl-1H-pyrrol-3-ylmethyl)-(1H-benzoimidazol-2-ylmethyl)-(5,6,7,8-tetrahydro-quinolin-8-yl)-amine;
(4-Aminomethyl-1H-pyrazol-3-ylmethyl)-(1H-benzoimidazol-2-ylmethyl)-(5,6,7,8-tetrahydro-quinolin-8-yl)-amine;
(4-Aminomethyl-1-methyl-1H-pyrazol-3-ylmethyl)-(1H-benzoimidazol-2-ylmethyl)-(5,6,7,8-tetrahydro-quinolin-8-yl)-amine;
(3-Aminomethyl-1H-pyrazol-4-ylmethyl)-(1H-benzoimidazol-2-ylmethyl)-(5,6,7,8-tetrahydro-quinolin-8-yl)-amine;
(3-Aminomethyl-1-methyl-1H-pyrazol-4-ylmethyl)-(1H-benzoimidazol-2-ylmethyl)-(5,6,7,8-tetrahydro-quinolin-8-yl)-amine;
(5-Aminomethyl-3H-imidazol-4-ylmethyl)-(1H-benzoimidazol-2-ylmethyl)-(5,6,7,8-tetrahydro-quinolin-8-yl)-amine;
(5-Aminomethyl-1-methyl-1H-imidazol-4-ylmethyl)-(1H-benzoimidazol-2-ylmethyl)-(5,6,7,8-tetrahydro-quinolin-8-yl)-amine;
(5-Aminomethyl-thiazol-4-ylmethyl)-(1H-benzoimidazol-2-ylmethyl)-(5,6,7,8-tetrahydro-quinolin-8-yl)-amine;
(5-Aminomethyl-pyrimidin-4-ylmethyl)-(1H-benzoimidazol-2-ylmethyl)-(5,6,7,8-tetrahydro-quinolin-8-yl)-amine;
(5-Aminomethyl-pyridazin-4-ylmethyl)-(1H-benzoimidazol-2-ylmethyl)-(5,6,7,8-tetrahydro-quinolin-8-yl)-amine;
(5-Allylaminomethyl-2-{[(1H-benzoimidazol-2-ylmethyl)-(5,6,7,8-tetrahydro-quinolin-8-yl)-amino]-methyl}-phenyl)-methanol;
(3-Allylaminomethyl-4-{[(1H-benzoimidazol-2-ylmethyl)-(5,6,7,8-tetrahydro-quinolin-8-yl)-amino]-methyl}-phenyl)-methanol;
(4-Allylaminomethyl-2-methoxymethyl-benzyl)-(1H-benzoimidazol-2-ylmethyl)-(5,6,7,8-tetrahydro-quinolin-8-yl)-amine;
(3-Allylaminomethyl-4-methoxymethyl-benzyl)-(1H-benzoimidazol-2-ylmethyl)-(5,6,7,8-tetrahydro-quinolin-8-yl)-amine;
(2-{[(1H-Benzoimidazol-2-ylmethyl)-(5,6,7,8-tetrahydro-quinolin-8-yl)-amino]-methyl}-5-cyclopropylaminomethyl-phenyl)-methanol;
(4-{[(1H-Benzoimidazol-2-ylmethyl)-(5,6,7,8-tetrahydro-quinolin-8-yl)-amino]-methyl}-3-cyclopropylaminomethyl-phenyl)-methanol;
(1H-Benzoimidazol-2-ylmethyl)-(4-cyclopropylaminomethyl-2-methoxymethyl-benzyl)-(5,6,7,8-tetrahydro-quinolin-8-yl)-amine;
(1H-Benzoimidazol-2-ylmethyl)-(2-cyclopropylaminomethyl-4-methoxymethyl-benzyl)-(5,6,7,8-tetrahydro-quinolin-8-yl)-amine;
5-Aminomethyl-2-{[(1H-benzoimidazol-2-ylmethyl)-(5,6,7,8-tetrahydro-quinolin-8-yl)-amino]-methyl}-benzamide;
5-Aminomethyl-2-{[(1H-benzoimidazol-2-ylmethyl)-(5,6,7,8-tetrahydro-quinolin-8-yl)-amino]-methyl}-N-hydroxy-benzamide;
5-Aminomethyl-2-{[(1H-benzoimidazol-2-ylmethyl)-(5,6,7,8-tetrahydro-quinolin-8-yl)-amino]-methyl}-benzoic acid hydrazide;
5-Aminomethyl-2-{[(1H-benzoimidazol-2-ylmethyl)-(5,6,7,8-tetrahydro-quinolin-8-yl)-amino]-methyl}-benzoic acid;
(1H-Benzoimidazol-2-ylmethyl)-(2,4-bis-allylaminomethyl-benzyl)-(5,6,7,8-tetrahydro-quinolin-8-yl)-amine;
(4-Allylaminomethyl-2-cyclopropylaminomethyl-benzyl)-(1H-benzoimidazol-2-ylmethyl)-(5,6,7,8-tetrahydro-quinolin-8-yl)-amine;
(2-Allylaminomethyl-4-cyclopropylaminomethyl-benzyl)-(1H-benzoimidazol-2-ylmethyl)-(5,6,7,8-tetrahydro-quinolin-8-yl)-amine;
(1H-Benzoimidazol-2-ylmethyl)-(2,4-bis-cyclopropylaminomethyl-benzyl)-(5,6,7,8-tetrahydro-quinolin-8-yl)-amine;
(2-Aminomethyl-4-propyl-benzyl)-(1H-benzoimidazol-2-ylmethyl)-(5,6,7,8-tetrahydro-quinolin-8-yl)-amine;
(4-Allyl-2-aminomethyl-benzyl)-(1H-benzoimidazol-2-ylmethyl)-(5,6,7,8-tetrahydro-quinolin-8-yl)-amine;
Acetic acid 3-aminomethyl-4-{[(1H-benzoimidazol-2-ylmethyl)-(5,6,7,8-tetrahydro-quinolin-8-yl)-amino]-methyl}-benzyl ester;
Acetic acid 5-aminomethyl-2-{[(1H-benzoimidazol-2-ylmethyl)-(5,6,7,8-tetrahydro-quinolin-8-yl)-amino]-methyl}-benzyl ester;
Acetic acid 4-{[(1H-benzoimidazol-2-ylmethyl)-(5,6,7,8-tetrahydro-quinolin-8-yl)-amino]-methyl}-3-cyclopropylaminomethyl-benzyl ester;
Acetic acid 2-{[(1H-benzoimidazol-2-ylmethyl)-(5,6,7,8-tetrahydro-quinolin-8-yl)-amino]-methyl}-5-cyclopropylaminomethyl-benzyl ester;
Acetic acid 3-allylaminomethyl-4-{[(1H-benzoimidazol-2-ylmethyl)-(5,6,7,8-tetrahydro-quinolin-8-yl)-amino]-methyl}-benzyl ester;
Acetic acid 5-allylaminomethyl-2-{[(1H-benzoimidazol-2-ylmethyl)-(5,6,7,8-tetrahydro-quinolin-8-yl)-amino]-methyl}-benzyl ester;
5-Aminomethyl-2-{[(1H-benzoimidazol-2-ylmethyl)-(5,6,7,8-tetrahydro-quinolin-8-yl)-amino]-methyl}-benzaldehyde oxime;
3-Aminomethyl-4-{[(1H-benzoimidazol-2-ylmethyl)-(5,6,7,8-tetrahydro-quinolin-8-yl)-amino]-methyl}-benzaldehyde oxime;
N-(5-Aminomethyl-2-{[(1H-benzoimidazol-2-ylmethyl)-(5,6,7,8-tetrahydro-quinolin-8-yl)-amino]-methyl}-benzyl)-acetamide;
N-(3-Aminomethyl-4-{[(1H-benzoimidazol-2-ylmethyl)-(5,6,7,8-tetrahydro-quinolin-8-yl)-amino]-methyl}-benzyl)-acetamide;
N-(3-(Acetylamino-methyl)-4-{[(1H-benzoimidazol-2-ylmethyl)-(5,6,7,8-tetrahydro-quinolin-8-yl)-amino]-methyl}-benzyl)-acetamide;
N-(2-{[(1H-Benzoimidazol-2-ylmethyl)-(5,6,7,8-tetrahydro-quinolin-8-yl)-amino]-methyl}-benzyl)-acetamide;
(6-Aminomethyl-1,3-dihydro-isobenzofuran-5-ylmethyl)-(1H-benzoimidazol-2-ylmethyl)-(5,6,7,8-tetrahydro-quinolin-8-yl)-amine;
(4-Aminomethyl-1,3-dihydro-isobenzofuran-5-ylmethyl)-(1H-benzoimidazol-2-ylmethyl)-(5,6,7,8-tetrahydro-quinolin-8-yl)-amine;
(7-Aminomethyl-1,3-dihydro-isobenzofuran-4-ylmethyl)-(1H-benzoimidazol-2-ylmethyl)-(5,6,7,8-tetrahydro-quinolin-8-yl)-amine;
N′-(1H-benzimidazol-2-ylmethyl)-N′-(5,6,7,8-tetrahydro-8-quinolinyl)-1,3-benzenedimethanamine;
(1H-Benzimidazol-2-ylmethyl)-(2-Aminomethyl-benzyl)-(5,6,7,8-tetrahydro-quinolin-8-yl)-amine (hydrobromide salt);
(2-Aminomethyl-benzyl)-(1H-benzimidazol-2-ylmethyl)-(5)-5,6,7,8-tetrahydro-quinolin-8-yl-amine (hydrochloride salt);
(3-aminomethyl-4-{[(1H-benzoimidazol-2-ylmethyl)-(5,6,7,8-tetrahydro-quinolin-8-yl)-amino]-methyl}-phenyl)-methanol;
(2-Aminomethyl-3-methoxy-benzyl)-(1H-benzimidazol-2-ylmethyl)-(5,6,7,8-tetrahydro-quinolin-8-yl)-amine(hydrobromide salt);
(1H-Benzimidazol-2-ylmethyl)-(5,6,7,8-tetrahydroquinolin-8-yl)-[(1-aminomethyl)-benzoxazol-3-ylmethyl)]-amine (hydrobromide salt);
(1H-Benzimidazol-2-ylmethyl)-(5,6,7,8-tetrahydroquinolin-8-yl)-[(1-benzyl-2-aminomethyl)-imidazol-5-ylmethyl)]-amine;
6-aminomethylpyridin-3-ylmethyl-(1H-benzimidazol-2-ylmethyl)-(5,6,7,8-tetrahydroquinolin-8-yl)-amine;
[4-(2-amino-ethyl)-benzyl]-(1H-benzoimidazol-2-ylmethyl)-(5,6,7,8-tetrahydro-quinolin-8-yl)-amine (hydrobromide salt);
[4-(3-amino-propyl)-benzyl]-(1H-benzoimidazol-2-ylmethyl)-(5,6,7,8-tetrahydro-quinolin-8-yl)-amine (hydrobromide salt);
N-(4-{[(1H-benzimidazol-2-ylmethyl)-(5,6,7,8-tetrahydro-quinolin-8-yl)-amino]-methyl}-benzyl)-hydroxylamine;
(5-aminomethyl-2-{[(1H-benzoimidazol-2-ylmethyl)-(5,6,7,8-tetrahydro-quinolin-8-yl)-amino]-methyl}-phenyl)-methanol;
2-Aminomethyl-5-{[(1H-benzimidazol-2-ylmethyl)-(5,6,7,8-tetrahydro-quinolin-8-yl)-amino]-methyl}-phenol (hydrobromide salt);
(4-Aminomethyl-3-methoxy-benzyl)-(1H-benzimidazol-2-ylmethyl)-(5,6,7,8-tetrahydro-quinolin-8-yl)-amine (hydrobromide salt);
(1H-benzoimidazol-2-ylmethyl)-(2,4-bis-aminomethyl-benzyl)-(5,6,7,8-tetrahydro-quinolin-8-yl)-amine (hydrobromide salt);
5-Aminomethyl-2-{[(1H-benzoimidazol-2-ylmethyl)-(5,6,7,8-tetrahydro-quinolin-8-yl)-amino]-methyl}-benzoic acid methyl ester (hydrobromide salt);
3-aminomethyl-4-{[(1H-benzimidazole-2-ylmethyl)-(5,6,7,8-tetrahydro-quinolin-8-yl)-amino]-methyl}-benzoic acid hydrobromide salt;
3-aminomethyl-4-{[(1H-benzimidazole-2-ylmethyl)-(5,6,7,8-tetrahydro-quinolin-8-yl)-amino]-methyl}-N-hydroxy-benzamide hydrobromide salt;
3-aminomethyl-4-{[(1H-benzimidazole-2-ylmethyl)-(5,6,7,8-tetrahydro-quinolin-8-yl)-amino]-methyl}-benzamide hydrobromide salt;
3-Aminomethyl-4-{[(1H-benzimidazole-2-ylmethyl)-(5,6,7,8-tetrahydro-quinolin-8-yl)-amino]-methyl}-benzoic acid hydrazide (hydrobromide salt);
(2-aminomethyl-5-fluorobenzyl)-(1H-benzimidazol-2-ylmethyl)-(5,6,7,8-tetrahydro-quinolin-8-yl)-amine (hydrobromide salt);
3-aminomethyl-4-{[(1H-benzimidazol-2-ylmethyl)-(5,6,7,8-tetrahydro-quinolin-8-yl)-amino]-methyl}-benzoic acid methyl ester;
(2-aminomethyl-4-methoxymethyl-benzyl)-(1H-benzimidazol-2-ylmethyl)-(5,6,7,8-tetrahydro-quinolin-8-yl)-amine;
N-(2-{[(1H-benzimidazol-2-ylmethyl)-(5,6,7,8-tetrahydroquinolin-8-yl)-amino]-methyl}-benzyl)-guanidine;
N-(4-{[(1H-benzimidazol-2-ylmethyl)-(5,6,7,8-tetrahydroquinolin-8-yl)-amino]-methyl}-benzyl)-guanidine (hydrobromide salt);
N′-(4-{[(1H-Benzimidazol-2-ylmethyl)-(5,6,7,8-tetrahydro-quinolin-8-yl)-amino]-methyl}-benzyl)-N,N-dimethyl-guanidine (hydrobromide salt);
[4-(1H-benzimidazol-2-ylmethyl)-(5,6,7,8-tetrahydroquinolin-8-yl)-aminomethylbenzyl]-N,N-dimethylformamidine (hydrobromide salt);
N-(4-{[(1H-Benzimidazol-2-ylmethyl)-(5,6,7,8-tetrahydro-quinolin-8-yl)-amino]-methyl}-benzyl)-benzamidine (hydrobromide salt);
N-(4-{[(1H-Benzimidazol-2-ylmethyl)-(5,6,7,8-tetrahydro-quinolin-8-yl)-amino]-methyl}-benzyl)-acetamidine (hydrobromide salt);
N-isobutyl-N′-(1H-benzimidazol-2-ylmethyl)-N′-(5,6,7,8-tetrahydro-8-quinolinyl)-1,4-benzenedimethanamine (hydrobromide salt);
(1H-Benzimidazol-2-ylmethyl)-(4-piperidin-2-yl-benzyl)-(5,6,7,8-tetrahydro-quinolin-8-yl)-amine (hydrobromide salt);
(1H-Benzimidazol-2-ylmethyl)-(4-piperidin-1-ylmethyl-benzyl)-(5,6,7,8-tetrahydro-quinolin-8-yl)-amine (hydrobromide salt);
(1H-Benzimidazol-2-ylmethyl)-(4-methylaminomethyl-benzyl)-(5,6,7,8-tetrahydro-quinolin-8-yl)-amine (hydrobromide salt);
(1H-Benzimidazol-2-ylmethyl)-(4-piperazin-1-ylmethyl-benzyl)-(5,6,7,8-tetrahydro-quinolin-8-yl)-amine (hydrobromide salt);
[4-(4-Allyl-piperazin-1-ylmethyl)-benzyl]-(1H-benzimidazol-2-ylmethyl)-(5,6,7,8-tetrahydro-quinolin-8-yl)-amine (hydrobromide salt);
(1H-Benzimidazol-2-ylmethyl)-(4-dimethylaminomethyl-benzyl)-(5,6,7,8-tetrahydro-quinolin-8-yl)-amine (hydrobromide salt);
(1H-Benzimidazol-2-ylmethyl)-(5,6,7,8-tetrahydro-quinolin-8-yl)-[4-(1,2,4-triazol-4-yliminomethyl)-benzyl]-amine (hydrobromide salt);
N′-(4-{[(1H-Benzimidazol-2-ylmethyl)-(5,6,7,8-tetrahydro-quinolin-8-yl)-amino]-methyl}-benzyl)-ethane-1,2-diamine (hydrobromide salt);
(1H-benzimidazol-2-ylmethyl)-(4-butylaminomethyl-benzyl)-(5,6,7,8-tetrahydro-quinolin-8-yl)-amine;
(1H-benzimidazol-2-ylmethyl)-(4-diallylaminomethyl-benzyl)-(5,6,7,8-tetrahydro-quinolin-8-yl)-amine;
(4-allylaminomethyl-benzyl)-(1H-benzimidazol-2-ylmethyl)-(5,6,7,8-tetrahydro-quinolin-8-yl)-amine;
(1H-benzimidazol-2-ylmethyl)-(4-pyrrolidin-1-ylmethyl-benzyl)-(5,6,7,8-tetrahydro-quinolin-8-yl)-amine;
(1H-Benzimidazol-2-ylmethyl)-(4-morpholin-4-ylmethyl-benzyl)-(5,6,7,8-tetrahydro-quinolin-8-yl)-amine (hydrobromide salt);
(1H-benzoimidazol-2-ylmethyl)-(5,6,7,8-tetrahydro-quinolin-8-yl)-(4-thiomorpholin-4-ylmethyl-benzyl)-amine;
(1H-Benzimidazol-2-ylmethyl)-(5,6,7,8-tetrahydro-quinolin-8-yl)-(2-cyclopropylaminomethyl-benzyl)-amine (HBr salt);
(1H-Benzimidazol-2-ylmethyl)-(5,6,7,8-tetrahydro-quinolin-8-yl)-(2-allylaminomethyl-benzyl)-amine (HBr salt);
(1H-Benzimidazol-2-ylmethyl)-[2-(R)-(2-aminopropionamidylmethyl)-benzyl]-(5,6,7,8-tetrahydro-quinolin-8-yl)-amine (hydrobromide salt);
(1H-benzimidazol-2-ylmethyl)-[2-(1H-benzimidazol-2-ylmethyl)-aminobenzyl]-(5,6,7,8-tetrahydroquinolin-8-yl)-amine;
(2-aminobenzyl)-(1H-benzimidazol-2-ylmethyl)-(5,6,7,8-tetrahydroquinolin-8-yl)-amine;
(1H-Benzimidazol-2-ylmethyl)-(2-cyano-benzyl)-(5,6,7,8-tetrahydro-quinolin-8-yl)-amine (hydrobromide salt);
2-{[(1H-Benzimidazol-2-ylmethyl)-(5,6,7,8-tetrahydro-quinolin-8-yl)-amino]-methyl}-6-methoxy-benzoic acid ethyl ester (hydrobromide salt);
(6-aminopyridin-3-ylmethyl)-(benzimidazol-2-ylmethyl)-(5,6,7,8-tetrahydroquinolin-8-yl)-amine (hydrobromide salt);
(2-aminopyridin-3-ylmethyl)-(benzimidazol-2-ylmethyl)-(5,6,7,8-tetrahydro-8-quinolinyl)-amine (hydrobromide salt);
N-(4-{[(1H-Benzimidazol-2-ylmethyl)-(5,6,7,8-tetrahydro-quinolin-8-yl)-amino]-methyl}-phenyl)-guanidine (hydrobromide salt);
(4-Amino-benzyl)-(1H-benzoimidazol-2-ylmethyl)-(5,6,7,8-tetrahydro-quinolin-8-yl)-amine (hydrobromide salt);
N′-({[(1H-Benzimidazol-2-ylmethyl)-(5,6,7,8-tetrahydroquinolin-8-yl)-amino]-methyl}-phenyl)-N,N-dimethylformamidine;
4-{[(1H-Benzimidazol-2-ylmethyl)-(5,6,7,8-tetrahydro-quinolin-8-yl)-amino]-methyl}-benzaldehyde oxime;
[4-(1H-benzimidazol-2-ylmethyl)-(5,6,7,8-tetrahydroquinolin-8-yl)-aminomethyl]-benzamidine (hydrobromide salt);
4-{[(1H-Benzimidazol-2-ylmethyl)-(5,6,7,8-tetrahydro-quinolin-8-yl)-amino]-methyl}-benzyl alcohol;
4-{[(1H-Benzimidazol-2-ylmethyl)-(5,6,7,8-tetrahydro-quinolin-8-yl)-amino]-methyl}-benzaldehyde;
4-{[(1H-Benzimidazol-2-ylmethyl)-(5,6,7,8-tetrahydro-quinolin-8-yl)-amino]-methyl}-benzoic acid methyl ester;
(R,S)-4-{[(1H-Benzimidazol-2-ylmethyl)-(5,6,7,8-tetrahydro-quinolin-8-yl)-amino]-methyl}-N-hydroxy-benzamide;
4-{[(1H-Benzimidazol-2-ylmethyl)-(5,6,7,8-tetrahydro-quinolin-8-yl)-amino]-methyl}-benzoic acid hydrazide;
4-{[(1H-benzoimidazol-2-ylmethyl)-(5,6,7,8-tetrahydro-quinolin-8-yl)-amino]-methyl}-benzoic acid (hydrobromide salt);
4-{[(1H-Benzimidazol-2-ylmethyl)-(5,6,7,8-tetrahydro-quinolin-8-yl)-amino]-methyl}-benzamide;
(6-Amino-pyridin-2-ylmethyl)-(1H-benzimidazol-2-ylmethyl)-(5,6,7,8-tetrahydro-quinolin-8-yl)-amine (hydrobromide salt);
(2-{[(1H-Benzimidazol-2-ylmethyl)-(5,6,7,8-tetrahydro-quinolin-8-yl)-amino]-methyl}-phenyl)-methanol (free base);
O-(2-{[(1H-Benzimidazol-2-ylmethyl)-(5,6,7,8-tetrahydro-quinolin-8-yl)-amino]-methyl}-benzyl)-hydroxylamine (hydrobromide salt);
(4-Amino-pyridin-3-ylmethyl)-(1H-benzimidazol-2-ylmethyl)-(5,6,7,8-tetrahydro-quinolin-8-yl)-amine (hydrobromide salt);
2-{[(1H-Benzimidazol-2-ylmethyl)-(5,6,7,8-tetrahydro-quinolin-8-yl)-amino]-methyl}-5-cyano-benzoic acid methyl ester;
4-{[(1H-benzimidazol-2-ylmethyl)-(5,6,7,8-tetrahydroquinolin-8-yl)-amino]-methyl}-3-cyano-benzamide;
[3-(1H-benzimidazol-2-yl)-benzyl]-(1H-benzimidazol-2-ylmethyl)-(5,6,7,8-tetrahydroquinolin-8-yl)-amine (hydrobromide salt);
(1H-Benzimidazol-2-ylmethyl)-(5,6,7,8-tetrahydroquinolin-8-yl)-(imidazol-2-yl)-methylamine (hydrobromide salt);
4-{[(1H-Benzimidazol-2-ylmethyl)-(5,6,7,8-tetrahydro-quinolin-8-yl)-amino]-methyl}-2,6-dichloropyridine (hydrobromide salt);
(1H-benzoimidazol-2-ylmethyl)-benzooxazol-5-ylmethyl-(5,6,7,8-tetrahydro-quinolin-8-yl)-amine;
pyridin-2-ylmethyl-(1H-benzimidazol-2-ylmethyl)-(5,6,7,8-tetrahydroquinolin-8-yl)-amine;
(1H-benzimidazol-2-ylmethyl)-benzoxazol-6-ylmethyl-(5,6,7,8-tetrahydro-quinolin-8-yl)-amine;
(1H-benzimidazol-4-ylmethyl)-(1H-benzimidazol-2-ylmethyl)-(5,6,7,8-tetrahydro-quinolin-8-yl)-amine (hydrobromide salt);
(1H-Benzimidazol-2-ylmethyl)-pyridin-4-ylmethyl-(5,6,7,8-tetrahydro-quinolin-8-yl)-amine;
(1H-Benzimidazol-2-ylmethyl)-(benzo[1,3]dioxol-4-ylmethyl)-(5,6,7,8-tetrahydro-quinolin-8-yl)-amine;
benzo[1,3]dioxol-5-ylmethyl-(1H-benzoimidazol-2-ylmethyl)-(5,6,7,8-tetrahydro-quinolin-8-yl)-amine;
(1H-Benzimidazol-2-ylmethyl)-(2,3-dihydro-benzofuran-7-ylmethyl)-(5,6,7,8-tetrahydro-quinolin-8-yl)-amine (hydrobromide salt);
(1H-Benzimidazol-2-ylmethyl)-pyridin-3-ylmethyl-(5,6,7,8-tetrahydro-quinolin-8-yl)-amine (hydrobromide salt);
(1H-benzoimidazol-5-ylmethyl)-(1H-benzoimidazol-2-ylmethyl)-(5,6,7,8-tetrahydro-quinolin-8-yl)-amine (hydrobromide salt);
Bis-(1H-benzimidazol-2-ylmethyl)-(5,6,7,8-tetrahydro-quinolin-8-yl)-amine (hydrobromide salt);
(1H-Benzimidazol-2-ylmethyl)-(3H-imidazol-4-ylmethyl)-(5,6,7,8-tetrahydro-quinolin-8-yl)-amine (hydrobromide salt);
[4-(1H-benzimidazol-2-yl)-benzyl]-(1H-benzimidazol-2-ylmethyl)-(5,6,7,8-tetrahydroquinolin-8-yl)-amine (hydrobromide salt);
(1H-Benzimidazol-2-ylmethyl)-(4-pyrid-2-yl-benzyl)-(5,6,7,8-tetrahydro-quinolin-8-yl)-amine (hydrobromide salt);
(1H-Benzimidazol-2-ylmethyl)-[4-(oxazol-2-yl)-benzyl]-(5,6,7,8-tetrahydroquinolin-8-yl)-amine (hydrobromide salt);
(1H-Benzimidazol-2-ylmethyl)-(4-imidazol-1-yl-benzyl)-(5,6,7,8-tetrahydro-quinolin-8-yl)-amine (hydrobromide salt);
[4-(thiazol-2-yl)-benzyl]-(1H-benzimidazol-2-ylmethyl)-(5,6,7,8-tetrahydroquinolin-8-yl)-amine (hydrobromide salt);
(1H-Benzimidazol-2-ylmethyl)[4-(benzothiazol-2-yl)-benzyl]-(5,6,7,8-tetrahydroquinolin-8-yl)-amine (hydrobromide salt);
[4-(benzoxazol-2-yl)-benzyl]-(1H-benzimidazol-2-ylmethyl)-(5,6,7,8-tetrahydroquinolin-8-yl)-amine (hydrobromide salt);
[4-(1H-imidazol-2-yl)-benzyl]-(1H-benzimidazol-2-ylmethyl)-(5,6,7,8-tetrahydroquinolin-8-yl)-amine (hydrobromide salt);
(2′-Aminomethyl-biphenyl-4-ylmethyl)-(1H-benzimidazol-2-ylmethyl)-(5,6,7,8-tetrahydro-quinolin-8-yl)-amine (hydrobromide salt);
(1H-Benzimidazol-2-ylmethyl)-(T-methoxy-biphenyl-4-ylmethyl)-(5,6,7,8-tetrahydro-quinolin-8-yl)-amine (hydrobromide salt);
(1H-Benzimidazol-2-ylmethyl)-(4-oxazol-5-yl-benzyl)-(5,6,7,8-tetrahydro-quinolin-8-yl)-amine (hydrobromide salt);
(1H-Benzimidazol-2-ylmethyl)-(5,6,7,8-tetrahydro-quinolin-8-yl)-(4-thiophen-2-yl-benzyl)-amine (hydrobromide salt);
(1H-Benzimidazol-2-ylmethyl)-[4-(2-methyl-2H-tetrazol-5-yl)-benzyl]-(5,6,7,8-tetrahydro-quinolin-8-yl)-amine (hydrobromide salt); and
(1H-Benzimidazol-2-ylmethyl)-[4-(5-phenyloxazol-2-yl)-benzyl]-(5,6,7,8-tetrahydroquinolin-8-yl)-amine.

Methods to synthesize the compounds useful in the method of the invention are set forth in the U.S. patents and applications incorporated hereinabove by reference.

As provided above, AMD3100 is an antagonist with the CXCR4 chemokine receptor (Gerlach, et al., J. Biol. Chem. (2001) 276:14153-14160). This compound interferes with the binding of bone marrow stromal cell derived SDF-1 with CXCR4 on stem cells which leads to the release of hematopoietic stem cells from bone marrow into the circulation (Broxmeyer, et al., Blood (2001) 98:811a (Abstract)). In a Phase 1 study at the University of Washington, Seattle, a single dose of 80 μg/kg of AMD-3100 resulted in a WBC count of 17,000/μl and a peak 6-fold increase in circulating CD34+ progenitor/stem cells at the 6 hour time point (Liles, et al., Blood 2001 98:737a (Abstract)).

In another recent study, mice were injected with rhG-CSF and recombinant rat Stem Cell Factor (rrSCF) in order to mobilize large numbers of bone marrow stem cells into the circulation and then we induced a heart attack. The combination of rrSCF and rhG-CSF provides a peak number of circulating stem cells after 5 daily injections. At 27 days post surgery there was a 68% improvement in survival in the treated group versus the controls. At this time the dead tissue was replaced with regenerating myocardium and all functional parameters tested were improved compared with controls (Orlic, et al., PNAS (2001) 98:10344-10349).

The compounds useful for the methods of the invention may be prepared in the form of prodrugs, i.e., protected forms which release the compounds of the invention after administration to the subject. Typically, the protecting groups are hydrolyzed in body fluids such as in the bloodstream thus releasing the active compound or are oxidized or reduced in vivo to release the active compound. A discussion of prodrugs is found in Smith and Williams Introduction to the Principles of Drug Design, Smith, H. J.; Wright, 2^{nd} ed., London (1988).

The polyamine compounds useful for the methods of the invention may be administered prepared in the forms of their acid addition salts or metal complexes thereof. Suitable acid addition salts include salts of inorganic acids that are biocompatible, including HCl, HBr, sulfuric, phosphoric and the like, as well as organic acids such as acetic, propionic, butyric and the like, as well as acids containing more than one carboxyl group, such as oxalic, glutaric, adipic and the like. Typically, at physiological pH, the compounds of the invention will be in the forms of the acid addition salts. Particularly preferred are the hydrochlorides. In addition, when prepared as purified forms, the compounds may also be crystallized as the hydrates.

The compounds useful for the methods of the invention may be administered as sole active ingredients, as mixtures of various compounds of formula (1), and/or in admixture with additional active ingredients that are therapeutically or nutritionally useful, such as antibiotics, vitamins, herbal extracts, anti-inflammatories, glucose, antipyretics, analgesics, granulocyte-macrophage colony stimulating factor (GM-CSF), Interleukin-1 (IL-1), Interleukin-3 (IL-3), Interleukin-8 (IL-8), PIXY-321 (GM-CSF/IL-3 fusion protein), macrophage inflammatory protein, stem cell factor, thrombopoietin, growth related oncogene or chemotherapy and the like.

The compounds useful for the methods of the invention may be formulated for administration to an animal subject using commonly understood formulation techniques well known in the art. Formulations which are suitable for particular modes of administration and for compounds of the type represented by those of formula (1) may be found in Remington's Pharmaceutical Sciences, latest edition, Mack Publishing Company, Easton, Pa.

In one embodiment, the compounds are administered by injection. For example, the compounds may be administered by intravenous, subcutaneous or intraperitoneal injection, and the like. Additional parenteral routes of administration include intramuscular and intraarticular injection. For intravenous or parenteral administration, the compounds are formulated in suitable liquid form with excipients as required. The compositions may contain liposomes or other suitable carriers. For injection intravenously, the solution is made isotonic using standard preparations such as Hank's solution.

Besides injection, other routes of administration may be used. The compounds may be formulated into tablets, capsules, syrups, powders, or other suitable forms for administration orally. By using suitable excipients, these compounds may also be administered through the mucosa using suppositories or intranasal sprays. Transdermal administration can also be effected by using suitable penetrants and controlling the rate of release.

The formulation and route of administration chosen will be tailored to the individual subject, the nature of the condition to be treated in the subject, and generally, the judgment of the attending practitioner.

Suitable dosage ranges for the compounds of formula (1) vary according to these considerations, but in general, the compounds are administered in the range of about 0.1 μg/kg-5 mg/kg of body weight; preferably the range is about 1 μg/kg-300 μg/kg of body weight; more preferably about 10 μg/kg-100 μg/kg of body weight. For a typical 70-kg human subject, thus, the dosage range is from about 0.7 μg-350 mg; preferably about 700 μg-21 mg; most preferably about 700 μg-7 mg. Dosages may be higher when the compounds are administered orally or transdermally as compared to, for example, i.v. administration.

The compounds may be administered as a single bolus dose, a dose over time, as in i.v. or transdermal administration, or in multiple dosages.

The compounds for use in the methods of the present invention may also be used in ex vivo treatment protocols to prepare cell cultures which are then directly administered to damaged tissues of the subjects. Ex vivo treatment can be conducted on autologous cells harvested from the peripheral blood or bone marrow or from allografts from matched donors. The concentration of the compound or compounds of formula (1)-(3), alone or in combination with other agents, such as macrophage inflammatory protein is a matter of routine optimization.

The present invention provides methods for regenerating tissue in a subject, comprising directly administering mobilized and harvested progenitor and/or stem cells to target damaged tissues in the subject. Various methods for direct administration to tissues are known in the art. For example, direct administration to tissues can be accomplished using catheter-based methods (e.g., infusion catheter, stiletto catheter, or balloon catheters), stents, needles, needle-free injectors, channeling devices, or other appropriate medical device for direct administration to a tissue. Alternatively, surgical approaches such as via open chest or thorascoscopy and surgical patches may be used for direct administration to a tissue, such as the myocardium.

Subjects that will respond favorably to the method of the invention include medical and veterinary subjects generally, including human patients. Among other subjects for whom the methods of the invention is useful are rats, mouse, cats, dogs, large animals, avians such as chickens, and the like. In general, any subject who would benefit from an elevation of progenitor cells and/or stem cells, or whose progenitor cells and/or stem cells are desirable for stem cell transplantation are appropriate for administration of the invention method.

In one embodiment, mobilized progenitor and/or stem cells are directly administered to damaged tissues in a subject who has suffered or is suffering from cardiovascular disease, arteriosclerosis, stroke, congestive heart failure, myocardial infarct, myocardial ischemia, or angina. The subject may also be a diabetic subject, who commonly suffer cardiovascular complications, including peripheral vascular diseases (Schatteman, et al., Journal of Clinical Investigation, 2000, vol., 106 no. 4, p. 571).

The damaged tissue may be a damaged organ tissue in the myocardium, limbs, brain, and liver. Other subjects that may benefit from the methods of the invention include subjects who have a hematopoietic disorder, such as aplastic anemia, leukemias, drug-induced anemias, and hematopoietic deficits from chemotherapy or radiation therapy.

The method of the invention is also useful in enhancing the success of transplantation during and following immunosuppressive treatments as well as in effecting more efficient wound healing and treatment of bacterial inflammation. The method of the present invention is further useful for treating subjects who are immunocompromised or whose immune system is otherwise impaired. Typical conditions which are ameliorated or otherwise benefited by the method of the present invention, include those subjects who are infected with a retrovirus and more specifically who are infected with human immunodeficiency virus (HIV). The method of the invention thus targets a broad spectrum of conditions for which elevation of progenitor cells and/or stem cells in a subject would be beneficial or, where harvesting of progenitor cells and/or stem cell for subsequent stem cell transplantation would be beneficial.

The methods of the invention may also be used to regenerate myocardium by mobilizing bone marrow stem cells. For example, harvested progenitor and/or stem cells are mobilized by compounds of formula (1)-(3), and directly administered to damaged myocardial tissue following myocardial infarct.

Having now generally described the invention, the same will be more readily understood through reference to the following examples which are provided by way of illustration, and are not intended to be limiting of the present invention, unless specified.

### Example 1

### Elevation of Mouse Progenitor Cell Levels

The effects of subcutaneous (s.c.) administration of 1,1′-[1,4-phenylene-bis(methylene)]-bis-1,4,8,11-tetraazacyclotetradecane (AMD3100) to C3H/H3 J mice on numbers of granulocyte macrophage (CFU-GM), erythroid (BFU-E), and multipotential (CFU-GEMM) progenitor cells per mL of blood were measured. Progenitors were stimulated to form colonies in vitro with the combination of 1 U/ml rhu Epo, 50 ng/ml rhu SLF, 5% ^{Vol}/_{Vol} pokeweed mitogen mouse spleen cell conditioned medium (PWMSCM), and 0.1 mM hemin Plates were scored 7 days after incubation.

The time dependent effects on the number of progenitors mobilized with AMD3100 are for a single s.c. injection of 5 mg/Kg and are shown in Table 1.

TABLE 1

| | Absolute Progenitors Per ML Blood | | |
|---|---|---|---|
| | Methylcellulose Culture | | |
| | CFU-GM | BFU-E | CFU-GEMM |
| Control | 289.8 | 49.4 | 25.8 |
| AMD3100: 15″ | 791.6 | 134.5 | 90.4 |
| AMD3100: 30″ | 1805.5 | 209.3 | 113.5 |
| AMD3100: 120″ | 828.7 | 102.3 | 47.6 |

To measure the dose-dependent effects, AMD3100 was administered at 1, 2.5, 5 and 10 mg/Kg via a single s.c. injection and the number of progenitors per mL of blood was measured at 1 hour post administration, and the results are shown in Table 2.

TABLE 2

| | Absolute Number Progenitors | | |
|---|---|---|---|
| | Per ML Blood Methylcellulose | | |
| | Culture | | |
| | CFU-GM | BFU-E | CFU-GEMM |
| Saline | 188.1 | 16 | 19 |
| AMD3100: 10 mg/kg | 825.6 | 120.5 | 79.8 |
| AMD3100: 5 mg/kg | 608.4 | 92.8 | 69.5 |
| AMD3100: 2.5 mg/kg | 687.6 | 98.9 | 70.6 |
| AMD3100: 1 mg/kg | 424 | 62 | 27.1 |

Fold Change Compared to Time 0

| | Progenitors | | |
|---|---|---|---|
| | Methylcellulose Culture | | |
| Time | GM | BFU-E | CFU-GEMM |
| 15″ | 2.73 | 2.72 | 3.51 |
| 30″ | 6.23 | 4.24 | 4.41 |
| 2′ | 2.86 | 2.07 | 1.85 |

Maximum mobilization of mouse progenitors is achieved at a dose of 2.5 to 10 mg/kg AMD3100, approximately 0.5 to 1 hour after injection, as shown in Table 3.

### Example 2

### Mobilization of Mouse Progenitor Cells in Combination with MIP-1α and G-CSF

The progenitor cell mobilization capacity of AMD3100 in combination with mouse (mu) macrophage inflammatory protein (MIP-1α) was tested with or without prior administration of rhu G-CSF. MIP-1α has been previously shown to mobilize progenitor cells in mice and humans (Broxmeyer, H. E., et al., Blood Cells, Molecules, and Diseases (1998) 24(2):14-30).

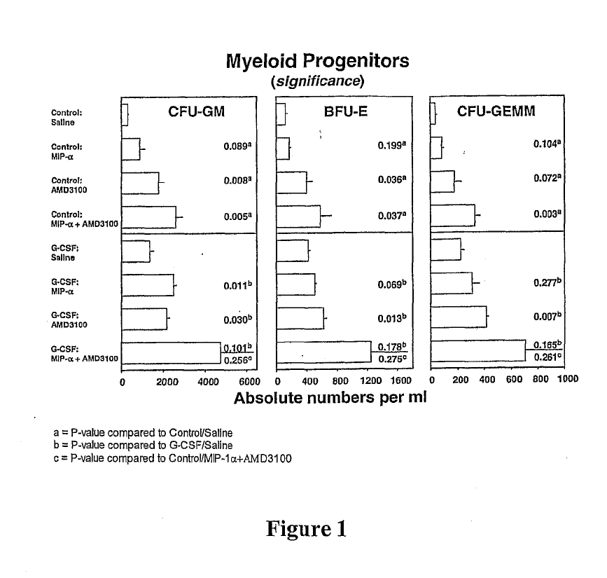

Groups of mice were randomized to receive control diluent (saline) or G-CSF at a dose of 2.5 μg per mouse, twice a day, for two days via s.c. injection. Eleven hours after the final injection of saline or G-CSF, the mice were divided into groups to receive MIP-1α administered I.V. at a total dose of 5 μg, AMD3100 administered s.c. at a dose of 5 mg/Kg, or a combination of both MIP-1α and AMD3100 at the same doses. One hour later, the mice were sacrificed and the number of progenitor cells per mL of blood were measured. These data are summarized in FIG. 1.

AMD3100 acts in an additive to greater than additive manner for mobilization of progenitor cells when used in combination with mouse (mu) macrophage inflammatory protein (MIP)-1α, each given 11 hours after the addition of rhu G-CSF or control diluent (saline) and 1 hour prior to assessing the blood.

### Example 3

### Clinical Elevation of Progenitor Cell Levels

Five healthy human volunteers having initial white blood cell counts of 4,500-7,500 cells/mm³ were used in the study. Each patient was given a single subcutaneous (s.c.) injection of 80 μg/kg AMD3100 (i.e., 1,1′-[1,4-phenylene-bis(methylene)]-bis-1,4,8,11-tetraazacyclotetradecane) in 0.9% saline, from a stock solution of 10 mg/mL AMD3100 in saline, under sterile conditions. Blood samples were obtained via catheter prior to the dose, and at various times up to 24 hours after dosing.

The blood samples were evaluated for total white blood cells, CD34 positive progenitor cells (via FACS analysis) as a percentage of total white blood cells, as well as the absolute numbers per mL and cycling status of granulocyte macrophage (CFU-GM), erythroid (BFU-E), and multipotential (CFU-GEMM) progenitor cells.

As shown in Tables 3 and 4, administration of AMD3100 caused an elevation of the white blood cell count and of CD34 positive progenitor cells in human volunteers which maximized at 6 hours post-administration.

TABLE 3
AMD3100 induced mobilization of white blood
cells in individual volunteers (×10³ WBC's).

| | | | TREATMENT | | | | | | |
|---|---|---|---|---|---|---|---|---|---|
| | | Base- | 30 | 1 | 2 | 4 | 6 | 9 | Day |
| ID | Screen | line | Min | Hr | Hr | Hr | Hr | Hr | 2 |
| P1 | 7.4 | 6.41 | 8.02 | 14.8 | 21.4 | 23.2 | 26.2 | 22.3 | 7.07 |
| P2 | 6.04 | 5.45 | 6.53 | 8.93 | 13.5 | 18.00 | 19.2 | 19.6 | 8.03 |
| P3 | 4.38 | 5.8 | 7.14 | 9.28 | ND | 18.10 | 17.9 | 18.4 | 4.98 |
| P4 | 5.08 | 5.31 | 4.37 | 7.38 | 12.4 | 14.6 | 15.8 | 13.9 | 4.98 |
| P5 | 4.53 | 5.02 | 6.08 | 8.43 | ND | 16.90 | 19.3 | 19.00 | 4.57 |

TABLE 4
AMD3100 induced mobilization of CD34 positive cells, expressed
as the percentage of the total WBC's in individual volunteers.

TREATMENT

| ID | Baseline | 1 Hr | 3 Hr | 6 Hr | 9 Hr | Day 2 |
|---|---|---|---|---|---|---|
| P1 | .07 | .04 | .07 | .11 | .11 | .08 |
| P2 | .08 | .06 | .08 | .13 | .11 | .12 |
| P3 | .07 | .16 | .06 | ND | .11 | .07 |
| P4 | .05 | .07 | .09 | .09 | .1 | .1 |
| P5 | .12 | .12 | .13 | .2 | .2 | .16 |

The blood was also analyzed for AMD3100 mobilized these progenitors.

Absolute numbers of unseparated and low density (Fico-hypaque separated) nucleated cells per ml of blood, as well as the absolute numbers per ml and cycling status of granulocyte macrophage (CFU-GM), erythroid (BFU-E), and multipotential (CFU-GEMM) progenitor cells were measured in normal donors injected s.c. with AMD3100. The above parameters were assessed prior to injection and at 1, 3, 6, 9 and 24 hours after injection of AMD3100. All progenitor cell results are based on the scoring of 3 culture plates per assay per point.

For the progenitor cell numbers and cycling status, the numbers of CFU-GM, BFU-E and CFU-GEMM in methylcellulose cultures by stimulation of the cells with 1 Unit (U)/ml recombinant human (rhu) erythropoietin, 100 U/ml rhu granulocyte-macrophage colony stimulating factor (GM-CSF), 100 U/ml rhu interleukin-3 (IL-3) and 50 ng/ml rhu steel factor (SLF=stem cell factor (SCF)). The CFU-GM were also evaluated in agar cultures stimulated with 100 U/ml rhu GM-CSF and 50 ng/ml rhu SLF. For both types of assays, colonies were scored after 14 day incubation in a humidified atmosphere with 5% CO₂ and lowered (5%) O₂ tension. Cell cycling status of progenitors was measured using a high specific activity tritiated thymidine kill technique as previously described (Broxmeyer, H. E., et al., Exp. Hematol. (1989) 17:455-459).

The results are given first, as the mean fold change in absolute numbers of nucleated cells and progenitors at 1, 3, 6, 9 and 24 hours compared to the preinjection (=Time (T) 0) counts for all five donors, as seen in Tables 5-7.

In the tables below,

STD—Standard deviation

STE—Standard error

PBL-US—peripheral blood-unseparated

PBL-LD—peripheral blood-low density (Ficoll Separated)

P—Significance using a 2 tailed t test

TABLE 5
Fold Change Compared to TIME = 0 (Average of 5 donors)

| | NUCLEATED CELLULARITY | | | | | | | | | |
|---|---|---|---|---|---|---|---|---|---|---|
| | PBL-US | | | | | PBL-LD | | | | |
| | MEAN | STD | STE | % CHG | P | MEAN | STD | STE | % CHG | P |
| T = 0 | 1.00 | 0.00 | 0.00 | 0.0% | | 1.00 | 0.00 | 0.00 | 0.0% | |
| T = 1 | 1.69 | 0.00 | 0.00 | 68.6% | 0.017 | 1.86 | 0.00 | 0.00 | 86.2% | 0.000 |
| T = 3 | 2.80 | 0.51 | 0.23 | 180.2% | 0.000 | 2.86 | 0.28 | 0.12 | 185.6% | 0.000 |
| T = 6 | 3.26 | 0.61 | 0.27 | 225.8% | 0.000 | 3.66 | 0.43 | 0.19 | 266.3% | 0.001 |
| T = 9 | 3.09 | 0.69 | 0.31 | 209.4% | 0.000 | 3.64 | 1.18 | 0.53 | 264.3% | 0.001 |
| T = 24 | 1.07 | 0.65 | 0.29 | 7.0% | 0.553 | 1.05 | 1.19 | 0.53 | 4.6% | 0.815 |

TABLE 6

| | METHYLCELLULOSE CULTURE | | | | | | | | | | | | | | |
|---|---|---|---|---|---|---|---|---|---|---|---|---|---|---|---|
| | CFU-GM | | | | | BFU-E | | | | | CFU-GEMM | | | | |
| | MEAN | STD | STE | % CHG | P | MEAN | STD | STE | % CHG | P | MEAN | STD | STE | % CHG | P |
| T = 0 | 1.00 | 0.00 | 0.00 | 0.0% | | 1.00 | 0.00 | 0.00 | 0.0% | | 1.00 | 0.00 | 0.00 | 0.0% | |
| T = 1 | 4.77 | 0.00 | 0.00 | 376.7% | 0.001 | 1.99 | 0.00 | 0.00 | 98.9% | 0.002 | 2.32 | 0.00 | 0.00 | 131.8% | 0.000 |
| T = 3 | 13.66 | 1.56 | 0.70 | 1266.5% | 0.001 | 3.21 | 0.50 | 0.22 | 221.3% | 0.004 | 4.33 | 0.44 | 0.20 | 332.5% | 0.000 |
| T = 6 | 21.71 | 5.78 | 2.58 | 2070.6% | 0.000 | 6.01 | 1.25 | 0.56 | 500.5% | 0.006 | 10.07 | 0.59 | 0.27 | 907.2% | 0.002 |
| T = 9 | 10.47 | 5.09 | 2.28 | 947.3% | 0.000 | 4.34 | 2.99 | 1.34 | 334.4% | 0.000 | 5.25 | 4.54 | 2.03 | 425.4% | 0.014 |
| T = 24 | 1.56 | 3.01 | 1.34 | 55.5% | 0.005 | 1.26 | 1.02 | 0.45 | 26.3% | 0.194 | 1.53 | 3.04 | 1.36 | 53.2% | 0.199 |

TABLE 7

| | AGAR CULTURE | | | | |
|---|---|---|---|---|---|
| | CFU-GM | | | | |
| | MEAN | STD | STE | % CHG | P |
| T = 0 | 1.00 | 0.00 | 0.00 | 0.0% | |
| T = 1 | 2.81 | 0.00 | 0.00 | 180.8% | 0.001 |
| T = 3 | 8.54 | 0.75 | 0.34 | 754.1% | 0.000 |
| T = 6 | 17.93 | 1.62 | 0.72 | 1692.8% | 0.000 |
| T = 9 | 10.25 | 4.57 | 2.04 | 924.9% | 0.000 |
| T = 24 | 2.08 | 2.06 | 1.03 | 108.3% | 0.073 |

The results are then shown as a fold change from T=0 levels for each individual donor, as shown in Tables 8-10.

TABLE 8
FOLD CHANGE COMPARED TO TIME = 0 for each individual patient (P)

| | NUCLEATED CELLULARITY | | | | | | | | | |
|---|---|---|---|---|---|---|---|---|---|---|
| | PBL-US | | | | | PBL-LD | | | | |
| | P1 | P2 | P3 | P4 | P5 | P1 | P2 | P3 | P4 | P5 |
| T = 0 | 1.00 | 1.00 | 1.00 | 1.00 | 1.00 | 1.00 | 1.00 | 1.00 | 1.00 | 1.00 |
| T = 1 | 2.54 | 1.38 | 1.38 | 1.36 | 1.76 | 2.07 | 1.99 | 1.48 | 1.66 | 2.10 |
| T = 3 | 3.55 | 2.74 | 2.02 | 2.46 | 3.23 | 2.83 | 3.25 | 2.17 | 2.82 | 3.20 |
| T = 6 | 3.97 | 2.94 | 2.74 | 2.60 | 4.04 | 4.07 | 3.90 | 2.27 | 2.78 | 5.30 |
| T = 9 | 3.27 | 3.30 | 2.69 | 2.24 | 3.96 | 3.65 | 4.43 | 2.47 | 2.48 | 5.17 |
| T = 24 | 1.21 | 1.43 | 0.96 | 0.77 | 0.99 | 1.01 | 1.71 | 0.79 | 0.60 | 1.12 |

TABLE 9
PROGENITORS

| | METHYLCELLULOSE CULTURE | | | | | | | | | | | | | | |
|---|---|---|---|---|---|---|---|---|---|---|---|---|---|---|---|
| | CFU-GM | | | | | BFU-E | | | | | CFU-GEMM | | | | |
| | P1 | P2 | P3 | P4 | P5 | P1 | P2 | P3 | P4 | P5 | P1 | P2 | P3 | P4 | P5 |
| T = 0 | 1.00 | 1.00 | 1.00 | 1.00 | 1.00 | 1.00 | 1.00 | 1.00 | 1.00 | 1.00 | 1.00 | 1.00 | 1.00 | 1.00 | 1.00 |
| T = 1 | 5.09 | 5.33 | 3.70 | 6.87 | 2.84 | 2.58 | 1.48 | 2.30 | 1.46 | 2.13 | 2.07 | 2.26 | 2.22 | 1.96 | 3.07 |
| T = 3 | 7.12 | 17.02 | 15.07 | 20.72 | 8.40 | 5.13 | 1.98 | 2.61 | 2.60 | 3.75 | 4.25 | 3.47 | 4.34 | 5.14 | 4.43 |
| T = 6 | 14.66 | 23.96 | 20.99 | 28.54 | 20.39 | 9.14 | 3.67 | 4.54 | 3.34 | 9.35 | 7.47 | 9.35 | 6.52 | 9.10 | 17.92 |
| T = 9 | 6.26 | 12.51 | 9.42 | 14.08 | 10.09 | 5.43 | 4.61 | 3.71 | 2.93 | 5.05 | 2.64 | 7.09 | 2.47 | 4.52 | 9.55 |
| T = 24 | 1.10 | 1.91 | 1.43 | 1.51 | 1.83 | 1.06 | 1.88 | 1.14 | 0.79 | 1.44 | 1.12 | 2.62 | 0.69 | 0.98 | 2.25 |

TABLE 10

| | AGAR CULTURE | | | | |
|---|---|---|---|---|---|
| | CFU-GM | | | | |
| | P1 | P2 | P3 | P4 | P5 |
| T = 0 | 1.00 | 1.00 | 1.00 | 1.00 | 1.00 |
| T = 1 | 3.05 | 3.74 | 1.67 | 2.71 | 2.87 |
| T = 3 | 8.88 | 9.49 | 7.47 | 10.46 | 6.40 |
| T = 6 | 17.77 | 24.01 | 14.04 | 13.07 | 20.75 |
| T = 9 | | 10.28 | 7.72 | 10.22 | 12.78 |
| T = 24 | | 3.69 | 1.13 | 1.30 | 2.20 |

The actual nucleated cell and progenitor cell numbers per ml of blood and the cycling status (=% progenitors in DNA synthesis (S) phase of the cell cycle) of progenitors for each of the five donors (#'s P1, P2, P3, P4, and P5) is shown in Tables 11 and 12.

TABLE 11

| | CFU-GM | | BFU-E | | CFU-GEMM | | CFU-GM | | BFU-E | | CFU-GEMM | |
|---|---|---|---|---|---|---|---|---|---|---|---|---|
| | P1 | | | | | | P2 | | | | | |
| | Absolute | | Absolute | | Absolute | | Absolute | | Absolute | | Absolute | |
| | # of | Cycling | # of | Cycling | # of | Cycling | # of | Cycling | # of | Cycling | # of | Cycling |
| | Pro- | Status | Pro- | Status | Pro- | Status | Pro- | Status | Pro- | Status | Pro- | Status |
| | genitors | of Pro- | genitors | of Pro- | genitors | of Pro- | genitors | of Pro- | genitors | of Pro- | genitors | of Pro- |
| | per ML | genitors | per ML | genitors | per ML | genitors | per ML | genitors | per ML | genitors | per ML | genitors |
| T = 0 | 247 | 6% | 261 | 0% | 127 | 6% | 273 | 0% | 410 | 2% | 120 | 0% |
| T = 1 | 1259 | 1% | 674 | 0% | 264 | 0% | 1455 | 0% | 608 | 3% | 272 | 6% |
| T = 3 | 1760 | 1% | 1340 | 13% | 540 | 7% | 4646 | 2% | 809 | 0% | 418 | 0% |
| T = 6 | 3624 | 0% | 2388 | 0% | 949 | 0% | 6540 | 0% | 1502 | 0% | 1126 | 0% |
| T = 9 | 1547 | 2% | 1418 | 11% | 335 | 0% | 3416 | 0% | 1886 | 0% | 854 | 4% |
| T = 24 | 271 | 0% | 278 | 0% | 142 | 0% | 521 | 3% | 768 | 2% | 316 | 0% |
| | CFU-GM | | BFU-E | | CFU-GEMM | | CFU-GM | | BFU-E | | CFU-GEMM | |
| | P3 | | | | | | P4 | | | | | |
| | Absolute | | Absolute | | Absolute | | Absolute | | Absolute | | Absolute | |
| | # of | Cycling | # of | Cycling | # of | Cycling | # of | Cycling | # of | Cycling | # of | Cycling |
| | Pro- | Status | Pro- | Status | Pro- | Status | Pro- | Status | Pro- | Status | Pro- | Status |
| | genitors | of Pro- | genitors | of Pro- | genitors | of Pro- | genitors | of Pro- | genitors | of Pro- | genitors | of Pro- |
| | per ML | genitors | per ML | genitors | per ML | genitors | per ML | genitors | per ML | genitors | per ML | genitors |
| T = 0 | 281 | 0% | 351 | 0% | 140 | 0% | 138 | 0% | 460 | 0% | 101 | 0% |
| T = 1 | 1040 | 0% | 806 | 0% | 312 | 0% | 947 | 0% | 672 | 0% | 199 | 0% |
| T = 3 | 4233 | 1% | 915 | 0% | 610 | 0% | 2857 | 5% | 1195 | 9% | 519 | 0% |
| T = 6 | 5895 | 0% | 1593 | 0% | 916 | 0% | 3936 | 0% | 1533 | 0% | 920 | 8% |
| T = 9 | 2647 | 0% | 1302 | 0% | 347 | 0% | 1942 | 0% | 1348 | 0% | 457 | 0% |
| T = 24 | 402 | 0% | 402 | 0% | 97 | 0% | 208 | 5% | 362 | 3% | 99 | 0% |

| | CFU-GM | | BFU-E | | CFU-GEMM | |
|---|---|---|---|---|---|---|
| | P5 | | | | | |
| | Absolute | | Absolute | | Absolute | |
| | # of | Cycling | # of | Cycling | # of | Cycling |
| | Pro- | Status | Pro- | Status | Pro- | Status |
| | genitors | of Pro- | genitors | of Pro- | genitors | of Pro- |
| | per ML | genitors | per ML | genitors | per ML | genitors |
| T = 0 | 169 | 0% | 343 | 1% | 55 | 0% |
| T = 1 | 481 | 0% | 730 | 0% | 169 | 0% |
| T = 3 | 1423 | 5% | 1288 | 3% | 244 | 0% |
| T = 6 | 3454 | 0% | 3208 | 1% | 987 | 0% |
| T = 9 | 1710 | 0% | 1731 | 0% | 526 | 0% |
| T = 24 | 310 | 0% | 495 | 0% | 124 | 0% |

TABLE 12

| | AGAR Culture | | AGAR Culture | | AGAR Culture | | AGAR Culture | | AGAR Culture | |
|---|---|---|---|---|---|---|---|---|---|---|
| | CFU-GM | | CFU-GM | | CFU-GM | | CFU-GM | | CFU-GM | |
| | P1 | | P2 | | P3 | | P4 | | P5 | |
| | Absolute | | Absolute | | Absolute | | Absolute | | Absolute | |
| | # of | Cycling | # of | Cycling | # of | Cycling | # of | Cycling | # of | Cycling |
| | Pro- | Status | Pro- | Status | Pro- | Status | Pro- | Status | Pro- | Status |
| | genitors | of Pro- | genitors | of Pro- | genitors | of Pro- | genitors | of Pro- | genitors | of Pro- |
| | per ML | genitors | per ML | genitors | per ML | genitors | per ML | genitors | per ML | genitors |
| T = 0 | 233 | 6% | 100 | 0% | 140 | 0% | 124 | 0% | 104 | 0% |
| T = 1 | 710 | 0% | 376 | 0% | 234 | 0% | 336 | 0% | 298 | 3% |
| T = 3 | 2070 | 0% | 953 | 1% | 1049 | 0% | 1299 | 0% | 664 | 0% |
| T = 6 | 4142 | 0% | 2409 | 3% | 1972 | 3% | 1623 | 0% | 2153 | 1% |
| T = 9 | | | 1032 | 0% | 1085 | 0% | 1268 | 0% | 1326 | 0% |
| T = 24 | | | 371 | 0% | 159 | 0% | 162 | 0% | 229 | 0% |

The results for all five donors were very consistent with maximal fold increases in circulating levels of progenitor cells seen 6 hours after injection of AMD3100 into the human donor subjects. Progenitors were in a slow or non-cycling state prior to and 1, 3, 6, 9 and 24 hours after injection of AMD3100.

### Example 4

### Mobilized Bone Marrow Stem Cells for Myocardial Repair

Adult rats are anesthetized and a thoracotomy is performed. The descending branch of the left coronary artery is ligated and not reperfused. Within 4 to 6 hours after ligation the animals are injected with limit dilution AMD-3100 or AMD-3100 plus rhG-CSF. Control rats are not treated with the reagents. The animals are monitored at one-week intervals by echocardiography and MRI. The experiment is terminated at 2, 6 to 12 weeks post-surgery. On the day of sacrifice, the hemodynamic functions are analyzed for left ventricle-end diastolic pressure, left ventricle-developed pressure and the rate of rise and fall of left ventricle pressure. The heart is then arrested in diastole and perfused via the abdominal aorta to flush residual blood from the vascular network of the myocardium. This is followed by perfusion of the heart with 10% formalin. Several slices are made through the fixed heart and these are embedded in paraffin and sections. The sections are stained and analyzed by light microscopy to determine the size of the infarct in the treated and control animals. Tissue sections from hearts taken at 2 weeks after surgery are stained with antibodies specific for immature, developing myocyte and blood vessel proteins and analyzed by confocal microscopy. The immunohistochemical analysis involves the identification of transcription factors and surface markers expressed in early stages of myocyte development. The results of this experiment will show that when the reagent AMD-3100 is administered within hours after induction of cardiac ischemia, together with or without rhG-CSF, this reagent mobilizes bone marrow stem cells rapidly, and will result in a block to cardiac remodeling and scar formation and will lead to regeneration of the dead myocardium.

### Example 5

### Tissue Regeneration in Diabetic Animal Ischemic Model

Diabetes was chemically induced in male nude Hfh11 mice using streptozotocin (STZ). Three to four weeks after injection of STZ the left hind limb was made ischemic by ligation of the iliac artery and removal of a 0.5-1 cm segment distal to the ligation.

AMD3100 was administered intraperitoneally with 5 mg/kg of a 1.25 mg/ml solution of AMD3100 (n=8) or an equal volume of normal saline (n=12) on the day of and two days after surgery. One group of additional mice (n=11) was injected intramuscularly into the ischemic hindlimb with human non-diabetic-derived unmobilized CD34+ (unCD34+) peripheral blood mononuclear cells (PBMCs), two to four hours after surgery. Human non-diabetic-derived unmobilized CD34+ are potent stimulators of vessel growth in diabetic mice, and may have similar properties in diabetic patients (Schatteman et al., J. Clin. Invest. (2000) 106:571-578). Another group of additional mice (n=7) was injected intramuscularly with AMD3100 mobilized CD34+ (mCD34+) human PBMCs (1×10⁶), which were harvested by apheresis from human volunteers four hours after subcutaneous injection with 240 ug/kg of AMD3100. Untreated mice served as controls (n=10).

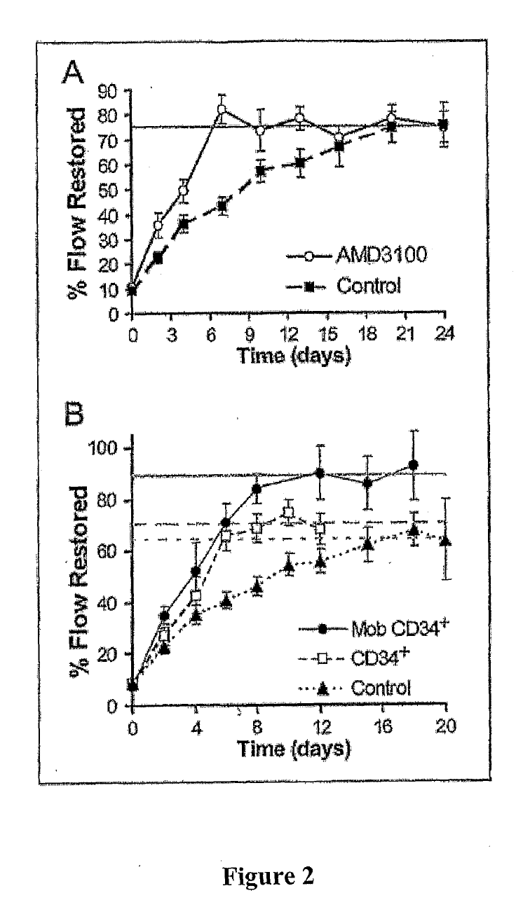

Limb blood flow restoration was analyzed immediately before and after surgery and at various times thereafter in the entire limb distal to the ligation using LASER Doppler analysis. FIG. 2A shows the percent of blood flow restored over time in mice injected intraperitoneally with AMD 3100 or vehicle (control) on days 0 and 2. Gray line indicates plateau of flow restoration (mean flow d8-d24) in AMD3100 treated mice. As shown in FIG. 2A, drug induced improvement was observed two days after surgery, and by seven days blood flow had reached its maximum in AMD3100 treated mice (FIG. 2A, P<0.05). Thereafter, blood flow was maintained at a mean of 74.6±2.8% that of the initial flow. Not until 16 days after surgery did flow in the control mice reach that of AMD3100 treated mice.

Furthermore, mCD34+ or unCD34+ PBMCs were injected directly into ischemic limb muscle to assess if AMD3100 mCD34+ PBMC therapy improves limb revascularization and if the cells are as potent as their unmobilized counterparts (i.e., unCD34+). FIG. 2B shows the percent of blood flow restored over time in mice injected with AMD3100 mobilized CD34+ or unmobilized CD34+ PBMCs into the ischemic muscle on the day of ligation. Gray lines indicate plateau of flow restoration using mean flow for d15-20 in untreated (lower, dotted), d8-d12 in unmobilized CD34+PBMC treated (middle, dashed), and d12-d18 in mobilized CD34+ cell treated (solid, upper) mice.

As shown in FIG. 2B, both mCD34+ and unCD34+ cell types significantly accelerated blood flow restoration relative to untreated controls by 6 days after injection (FIG. 2B, P<0.05). By 8 days after surgery blood flow values plateaued in unCD34+ PBMC injected limbs, maintaining a mean of 70.7±3.9% of initial flow thereafter. Flow in limbs of control mice plateaued at a similar level (64.7±4.6%) but did not reach this level until 15 days. In contrast, flow in mCD34+ PBMC treated limbs continued to improve until day 12, after which blood flow plateaued at 89.5±6.6% of initial flow, a flow that was significantly greater than that of either control or unCD34+ PBMC treated limbs.

The results demonstrate that systemic treatment with AMD3100 (FIG. 2A) and local injection of AMD3100 mCD34+ PBMCs (FIG. 2B) accelerate blood flow restoration to ischemic tissue in a diabetic environment with similar kinetics. AMD3100 systemic therapy accelerated blood flow restoration, but did not increase it. In contrast, mCD34+ PBMC therapy both accelerated and increased blood flow restoration, and only mCD34+ PBMCs increased the amount of flow covered in the ischemic limbs relative to untreated controls.

It is understood that the foregoing detailed description and accompanying examples are merely illustrative, and are not to be taken as limitations upon the scope of the invention. Various changes and modifications to the disclosed embodiments will be apparent to those skilled in the art, and may be made without departing from the spirit and scope thereof. U.S. patents and publications referenced herein are incorporated by reference.

1. A method for regenerating tissue in a subject, comprising directly administering mobilized and harvested progenitor and/or stem cells to damaged tissues in said subject, wherein the progenitor and/or stem are mobilized by a compound having formula (1)
Z-linker-Z′ (1)
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
or Z is of the formula 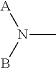
wherein A comprises a monocyclic or bicyclic fused ring system containing at least one N and B is H or an organic moiety of 1-20 atoms;
Z′ may be embodied in a form as defined by Z above, or alternatively may be of the formula
—N(R)—(CR₂)ₙ—X
wherein each R is independently H or straight, branched or cyclic alkyl (1-6C),
n is 1 or 2, and
X is an aromatic ring, including heteroaromatic rings, or is a mercaptan;
or Z¹ is of the formula —Ar(Y)ⱼ;
wherein Ar is an aromatic or heteroaromatic moiety, and each Y is independently a non-interfering substituent and j is 0-3;
“linker” represents a bond, alkylene (1-6C) or may comprise aryl, fused aryl, oxygen atoms contained in an alkylene chain, or may contain keto groups or nitrogen or sulfur atoms.

2. The method of claim 1, wherein said progenitor and/or stem cells are mobilized and harvested by apheresis.

3. The method of claim 1, wherein said progenitor and/or stem cells are mobilized into the peripheral blood or bone marrow of said subject and harvested.

4. The method of claim 3, wherein said peripheral blood or bone marrow is derived from a subject who has been treated with granulocyte colony-stimulating factor.

5. The method of claim 1, wherein said damaged tissues are ischemic tissue.

6. The method of claim 5 wherein said ischemic tissue is organ tissue or limb tissue.

7. The method of claim 1, wherein said damaged tissues are cardiac tissue, brain tissue, peripheral vascular tissue, hepatic tissue, renal tissue, gastrointestinal tissue, lung tissue, liver tissue, smooth muscle tissue, or striated muscle tissue.

8. The method of claim 1, wherein said compound of formula (1) is 1,1′-1,4-phenylene-bis-(methylene)-bis-1,4,8,11-tetraazacyclotetradecane (AMD 3100).

9. The method of claim 8, wherein AMD 3100 is in the form of a pharmaceutically acceptable salt.

10. The method of claim 9, wherein said pharmaceutically acceptable salt is an acid addition salt.

11. The method of claim 10, wherein the acid addition salt is hydrochloride.

12. The method of claim 1, wherein said compound of formula (1) is directly administered to damaged tissues in said subject in the dosage range of about 0.1 μg/kg-5 mg/kg of body weight.

13. The method of claim 12, wherein said compound of formula (1) is directly administered to damaged tissues in said subject in the dosage range of about 1 μg/kg to 300 μg/kg of body weight.

14. The method of claim 13, wherein said compound of formula (1) is directly administered to damaged tissues in said subject in the dosage range of about 10 μg/kg to 100 μg/kg of body weight.

15. The method of claim 1, wherein said progenitor and/or stem cells are harvested from bone marrow.

16. The method of claim 1, wherein the subject is human.

17. The method of claim 1, wherein said subject is diabetic, or is suffering or has suffered from limb ischemia or a peripheral vascular disease.

18. The method of claim 17, wherein said peripheral vascular disease is a peripheral arterial disease.

19. The method of claim 18, wherein said peripheral arterial disease is atherosclerosis, carotid artery disease, peripheral arterial disease of the legs, peripheral arterial disease of the renal arteries, abdominal aortic aneurysm, Raynaud syndrome, Buerger disease, or vasculitis.

20. The method of claim 1, wherein blood flow to said damaged tissues is increased and/or wherein damaged tissue is repaired.

21.-32. (canceled)

33. A method for increasing blood flow in a subject, comprising directly administering mobilized and harvested progenitor and/or stem cells to a target tissue in said subject that is in need of increased blood flow, wherein the progenitor and/or stem are mobilized by a compound having formula (1)
Z-linker-Z′ (1)
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
or Z is of the formula 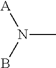
wherein A comprises a monocyclic or bicyclic fused ring system containing at least one N and B is H or an organic moiety of 1-20 atoms;
Z′ may be embodied in a form as defined by Z above, or alternatively may be of the formula
—N(R)—(CR₂)ₙ—X
wherein each R is independently H or straight, branched or cyclic alkyl (1-6C),
n is 1 or 2, and
X is an aromatic ring, including heteroaromatic rings, or is a mercaptan;
or Z¹ is of the formula —Ar(Y)ⱼ;
wherein Ar is an aromatic or heteroaromatic moiety, and each Y is independently a non-interfering substituent and j is 0-3;
“linker” represents a bond, alkylene (1-6C) or may comprise aryl, fused aryl, oxygen atoms contained in an alkylene chain, or may contain keto groups or nitrogen or sulfur atoms;
thereby increasing said blood flow in said tissue.

34. The use of a compound having formula (1) as defined in claim 1 for the manufacture of a medicament for tissue regeneration, wherein said compound of formula (1), is capable of mobilizing progenitor and/or stem cells, which are harvested and directly administered to damaged tissues in a subject.

35. The use according to claim 34, wherein said compound of formula (1) is AMD 3100.

36. The use of a compound having formula (1) for the manufacture of a medicament for increasing blood flow in a target tissue, wherein said compound having formula (1) is capable of mobilizing progenitor and/or stem cells, which are harvested and directly administered to target tissues in a subject.

37. The use according to claim 36, wherein said compound of formula (1) is AMD 3100.
